(19) United States
(12) Patent Application Publication
(10) Pub. No.: US 2013/0224109 A1
(43) Pub. Date: Aug. 29, 2013

(54) COMPOSITIONS AND METHODS FEATURING IL-6 AND IL-21 ANTAGONISTS

(71) Applicants: Terry Strom, Brookline, MA (US); Maria Koulmanda, Brookline, MA (US)

(72) Inventors: Terry Strom, Brookline, MA (US); Maria Koulmanda, Brookline, MA (US)

(73) Assignee: BETH ISRAEL DEACONESS MEDICAL CENTER, INC., BOSTON, MA (US)

(21) Appl. No.: 13/810,956

(22) PCT Filed: Jul. 20, 2011
(86) PCT No.: PCT/US11/44681

§ 371 Date: May 6, 2013

Related U.S. Application Data
(60) Provisional application No. 61/366,118, filed on Jul. 20, 2010.

(51) Int. Cl. A61K 39/395; C07K 16/46

(52) U.S. Cl. CPC A61K 39/3955; C07K 16/468
USPC 424/1.49; 530/387.3; 530/391.7; 530/391.3; 424/158.1; 424/172.1; 424/183.1; 424/178.1

(57) ABSTRACT

The present invention features compositions for inhibiting both the IL-6 and the IL-21 pathways and methods of making and using such compositions. Our work to date indicates the importance of the redundancy of IL-6 and IL-21 to perform certain crucial functions. The pathways can be inhibited by inhibiting the ligands (i.e., IL-6 and IL-21) and/or their respective receptors (i.e., the IL-6 receptor and IL-21 receptor). Alternatively, or in addition, upstream and downstream effectors in the IL-6 and IL-21 pathways can be blocked. The agents used can be antibody or antibody-based proteins or peptides including circulating receptors, optionally coupled to an immunoglobulin or a portion thereof (e.g., the Fc region). Also provided are methods for using the compositions, for example, in organ transplantation, tissue grafting, or autoimmune disorders.

### CROSS-REFERENCE TO RELATED APPLICATIONS

This application claims the benefit of the filing date of U.S. Provisional Application No. 61/366,118, which was filed on Jul. 20, 2010. For the purpose of any U.S. application that may claim the benefit of U.S. Provisional Application No. 61/366,118, the contents of that earlier filed application are hereby incorporated by reference in their entirety.

### FIELD OF THE INVENTION

The present invention relates to compositions and methods for modulating an immune response. More particularly, the invention encompasses methods of treating a patient who is receiving an organ transplant or suffering from an autoimmune disease by administering antagonists of IL-6 and IL-21.

### BACKGROUND

Heart transplantation is a life-saving procedure in patients with end-stage heart failure. Despite improved immunosuppressive regimens, many heart transplants undergo rejection and/or develop chronic vasculopathy, leading to the loss of graft function. According to the American Heart Association, about 85% of heart transplant recipients survive 1 year, 72% survive 5 years, about 50% survive 10 years and 16% survive 20 years after a heart transplant. Graft damage due to the rejection is the second major cause of death, after malignancies due to the chronic immunosuppression. These outcomes are simply unacceptable, particularly when the limited survival of pediatric transplant recipients is considered. Approximately 2,700 people are on the USA heart transplant list, and just over 2,100 heart transplants are performed yearly. Because of the small donor pool, re-transplantation is limited. Strategies that induce drug-free, graft tolerance would have a significant impact on patients' well-being and survival rates.

### SUMMARY OF THE INVENTION

The present invention features compositions for inhibiting both the IL-6 and the IL-21 pathways and methods of making and using such compositions. Our work to date indicates the importance of the redundancy of IL-6 and IL-21 to perform certain crucial functions. The pathways can be inhibited by inhibiting the ligands (i.e., IL-6 and IL-21) and/or their respective receptors (i.e., the IL-6 receptor and IL-21 receptor). Alternatively, or in addition, upstream and downstream effectors in the IL-6 and IL-21 pathways can be blocked. For example, one can use antisense, RNAi, or microRNA technologies to block the expression of components of the IL-6 and IL-21 pathways, including the ligands and receptors per se. The compositions (e.g., physiologically acceptable or pharmaceutical compositions) can include two different agents, one targeting the IL-6 pathway (e.g., an IL-6 cytokine or cytokine receptor) and one (a “second” agent) targeting the IL-21 pathway. Thus, en toto the compositions target both the IL-6 and IL-21 pathways. The compositions can also include bispecific molecules having one portion that targets a component of the IL-6 pathway (e.g., IL-6 or its receptor) and one (a “second” portion) targeting the IL-21 pathway. Thus, the compositions can include two anti-cytokine agents, two anti-receptor agents or a combination of anti-cytokine and anti-receptor agents. The agents used can be antibody or antibody-based proteins or peptides including circulating receptors, optionally coupled to an immunoglobulin or a portion thereof (e.g., the Fc region). The agents can also be mutant cytokines (e.g., IL-6 or IL-21 cytokines that bind but do not activate their respective receptors). In other embodiments, the agents can be other peptides that are receptor blocking agents or small molecules that inhibit the cytokines or their receptors. The agents targeting receptors can be agents that simply inhibit receptor activity or that inhibit the receptor and kill the target cell. Cell killing can be promoted through the use of antibodies with an isotype that activates complement/Fc receptor bearing leukocytes. A toxin or a radioactive component (e.g., an isotope) can also be used to, kill target cells.

The treatment methods are targeted toward autoimmune disease, including multiple sclerosis, type 1 diabetes, inflammatory bowel disease, psoriasis, systemic lupus erythematosis, rheumatoid arthritis; toward patients receiving any cellular, tissue, or organ transplant; and toward patients suffering from an ischemia reperfusion injury or anoxia-hypoxia (as occurs, for example, in acute kidney injury (formerly acute tubular necrosis) or myocardial infarct.

More generally, the methods can be used to treat any patient who has RORγt positive cells or an increase in the expression or number of such cells (e.g., at the site of injury, site of disease, or site of tissue rejection). In some methods of the present invention, one can first determine whether a patient has elevated RORγt cells and then make a determination as to whether or not to treat the patient with one or more of the compositions described herein.

Accordingly, the present invention is based, in part, on our studies indicating that the combined inhibition or blockade of two inflammatory cytokines, IL-6 and IL-21, with redundant and detrimental effects upon anti-donor immunity, can direct the response to an allograft toward a preferential commitment to the tissue protective, regulatory T-cell phenotype. While the invention is not limited or defined by the cellular events underlying the prophylactic or therapeutic outcome, and while a reciprocal decrease in the generation of Th17 and Th1 cells may well be important, we expect treatments targeting both IL-6 and IL-21 simultaneously will foster regulatory type alloimmunity. We also expect that the combined blockade of the two cytokine pathways will provide synergistic benefits in the induction of tolerance. Our work extends studies documenting prolonged survival of IL-6-deficient cardiac allografts (Perkins et al.) by recognizing the combined impact of donor and/or recipient IL-6 and IL-21 deficiencies simultaneously.

Unlike most approaches aimed at prolonging engraftment, the present methods are not aimed at impairing T-cell activation or directly depleting immune cell populations. Instead, we believe the methods guide alloactivated T-cells into a tissue protective mode rather than a tissue destructive mode. We believe that graft protective immunity can be fostered by modifying the detrimental influence of certain inflammatory cytokines upon donor-reactive T-cells because inflammatory cytokines such as IL-6, which are abundant in the peri-transplant period, promote differentiation of graft-destructive effector/memory T-cells while impairing the generation and function of graft-protective Tregs. An immune response conducted in the absence of these inflammatory stimuli should reliably tilt toward the tissue protective mode. While Foxp3 is expressed transiently in newly activated human, not mouse, T-cells, the role of stably expressed Foxp3+CD4+T-cells is identical in mice and humans as loss of these cells results in overwhelming and lethal autoimmunity.

The studies we have already designed and our work with knock-out mice can be rapidly translated to studies in wild type mice, to large animal models and to clinical practice, as therapeutic molecules already exist and at least one useful antagonist is FDA approved. Animal models for evaluating the present compositions and methods are well known in the art. For example, one could use the non-human primate heart transplant model, either alone or with the addition of rapamycin. Rapamycin, unlike calcineurin inhibitors or corticosteroids, supports commitment to the regulatory T-cell phenotype. IL-6 blockade is now approved for clinical use. Incorporating IL-6 antagonists in the present compositions and methods should avoid lymphodepletion and immunosuppression.

Our strategy for the creation of tolerance to grafted tissue (e.g. allografts or xenografts) and for the treatment of autoimmunity is to manipulate the signals that T cells receive from cytokines present within the milieu in which antigen activation occurs. These cytokines are, in large measure, the environmental cues that direct T cell commitment to tissue destructive or protective phenotypes. While either the donor (or an organ, tissue, or cell harvested therefrom) or a recipient may be treated with either an IL-6 or IL-21 antagonist (or both), the methods can also be carried out such that the donor (or an organ, tissue, or cell harvested therefrom) is treated with an IL-6 antagonist while the recipient is treated with an IL-21 antagonist. We reasoned that the donor tissue would be the principal source of IL-6 because it is at risk from ischemia and reperfusion injury and from anoxic injury. Further, the inability of the recipient to respond to IL-21 could be important for permanent engraftment.

In one aspect, the invention features a bi-specific immunoglobulin that includes a first portion that specifically binds an IL-6 or an IL-6 receptor and a second portion that specifically binds an IL-21 or an IL-21 receptor. More specifically, the first portion specifically binds an IL-6 and the second portion specifically binds an IL-21; the first portion specifically binds an IL-6 receptor and the second portion specifically binds an IL-21 receptor; the first portion specifically binds an IL-6 and the second portion specifically binds an IL-21 receptor; or the first portion specifically binds an IL-6 receptor and the second portion specifically binds an IL-21. The bi-specific immunoglobulin can be a bi-specific monoclonal antibody or a biologically active variant thereof, a chemically linked F(ab′)₂ or a biologically active variant thereof, or a bi-specific T cell engager or a biologically active variant thereof. The immunoglobulin can be a human, humanized, or chimeric immunoglobulin. In any of the embodiments, the IL-6 can be a human IL-6, the IL-6 receptor can be a human IL-6 receptor, the IL-21 can be a human IL-21, and/or the IL-21 receptor can be a human IL-21 receptor. The immunoglobulin can further comprise a toxin or radioisotope (which may kill the cells it is brought into proximity with).

The immunoglobulin can further include a detectable label, and in any instance where a detectable label is incorporated, the label can be one that is used in performing positron-emission tomography (PET); used to perform SPECT imaging; used in magnetic resonance imaging; one that is detectable by X-ray; or one that is detectable by ultrasound. The detectable label can be a radiopaque or contrast agent (e.g., barium, diatrizoate, ethiodized oil, gallium citrate, iocarmic acid, iocetamic acid, iodamide, iodipamide, iodoxamic acid, iogulamide, iohexyl, iopamidol, iopanoic acid, ioprocemic acid, iosefamic acid, ioseric acid, iosulamide meglumine, iosemetic acid, iotasul, iotetric acid, iothalamic acid, iotroxic acid, ioxaglic acid, ioxotrizoic acid, ipodate, meglumine, metrizamide, metrizoate, propyliodone, or thallous chloride). Where the detectable label is a fluorescent label, it can be fluorescein isothiocyanate, rhodamine, phycoerytherin, phycocyanin, allophycocyanin, o-phthaldehyde or fluorescamine. Where the detectable label is a chemiluminescent compound, it can be luminol, isoluminol, an aromatic acridinium ester, an imidazole, an acridinium salt or an oxalate ester. Where the detectable label is a bioluminescent compound, it can be luciferin, luciferase or aequorin. Where the detectable label is detectable by ultrasound, it can be a liposome or dextran.

In another aspect, the invention features a pharmaceutical composition comprising a bi-specific immunoglobulin as described herein.

In another aspect, the invention features a substantially pure mutant IL-21 polypeptide that includes an amino acid sequence that is at least or about 85% (e.g., at least or about 90%, 95%, or 98%) identical to SEQ ID NO: 2. Functionally, the mutant IL-21 polypeptide can bind to the α subunit of the IL-21 receptor but lack or substantially lack an ability to bind to the γ subunit of the IL-21 receptor. The amino acid sequence can include an amino acid substitution relative to SEQ ID NO: 2. Alternatively, or in addition, the amino acid sequence can include one or more amino acid additions or deletions relative to SEQ ID NO: 2. In one embodiment, the mutation is between amino acids 133 and 152 of SEQ ID NO: 2 (e.g., at position 145, for example, a substitution mutation at position 145). Any of the substitutions can replace a wild type amino acid residue with an alanine residue. For example, the mutation at position 145 can be a substitution of alanine for glutamine. The mutant IL-21 polypeptide can further include a mutation at position 150 (e.g., a substitution mutation, e.g., a substitution of alanine for glutamine). Functionally, the mutant IL-21 polypeptide can inhibit cellular proliferation relative to that which would normally occur when wild-type IL-21 (SEQ ID NO: 2) specifically binds to an IL-21 receptor complex.

Any of the mutant IL-21 polypeptides can include an amino acid sequence that increases the circulating half-life of the mutant IL-21 polypeptide (e.g., an Fc region of an IgG molecule lacking an IgG variable region, which may be lytic or non-lytic).

In another aspect, the invention features nucleic acid molecules (e.g., isolated, purified, or substantially isolated or purified nucleic acid molecules) encoding the IL-21 mutant polypeptides described herein.

In another aspect, the invention features vectors (e.g., plasmids and viral vectors) that contain the nucleic acid molecules described herein.

In another aspect, the invention features cells (e.g., bacterial cells and mammalian cells) that include a mutant IL-21 polypeptide, a nucleic acid encoding such polypeptide, or a vector containing such nucleic acid. The cells may be maintained in cell or tissue culture or within a non-human transgenic animal.

In another aspect, the invention features compositions (e.g., a physiologically acceptable or pharmaceutical composition) that include a mutant IL-21 polypeptide as described herein, a nucleic acid molecule as described herein, a vector as described herein, or a cell as described herein. Any of these compositions can further include an antagonist of the IL-6 pathway.

In another aspect, the invention features compositions (e.g., a physiologically acceptable or pharmaceutical composition) that include first and second agents, wherein the first agent comprises an IL-6 pathway antagonist and the second agent comprises an IL-21 pathway antagonist. The first agent can include an anti-IL-6 antibody or a biologically active variant thereof, an anti-IL-6 receptor antibody or a biologically active variant thereof, a mutant IL-6, a soluble IL-6 receptor, optionally coupled to an immunoglobulin, or a small organic compound that blocks IL-6 or an IL-6 receptor. The mutant IL-6 may bind but not activate the corresponding IL-6 receptor, and the soluble IL-6 receptor may bind a corresponding IL-6, thereby interfering with its binding to IL-6 receptors expressed by the patient's cells. The first agent can further include a toxin, a radioisotope, or detectable label. In any of these compositions, the second agent can be or can include an anti-IL-21 antibody or a biologically active variant thereof, an anti-IL-21 receptor antibody or a biologically active variant thereof, a mutant IL-21, a soluble IL-21 receptor, optionally coupled to an immunoglobulin, or a small organic compound that blocks IL-21 or an IL-21 receptor. The mutant IL-21 may bind but not activate the corresponding IL-21 receptor, and the soluble IL-21 receptor may bind a corresponding IL-21. The second agent can further include a toxin, a radioisotope, or detectable label.

Any of the pharmaceutical compositions of the invention can be formulated for use in the preparation of a medicament, and particular uses are indicated below in the context of treatment.

In another aspect, the invention features methods of reducing the likelihood of graft rejection in a patient. The methods can include a step of administering to the patient a therapeutically effective amount of a pharmaceutical composition as described herein. The graft can be an allograft or xenograft and can be of an organ, such as a heart, kidney, liver or a lobe thereof, lung or a lobe thereof, pancreas or a portion thereof, bone marrow, cartilage, skin, a cornea, neuronal tissue, or muscle. The graft can also include a population of cells that do not define an intact organ. Transplanted cells can also include stem cells (e.g., mesenchymal stem cells, adult stem cells, or fetal stem cells). In one embodiment, the cells can be pancreatic islet cells.

In another aspect, the invention features methods of treating a patient who is suffering from an autoimmune disease. The methods can include the step of administering to the patient a therapeutically effective amount of a pharmaceutical composition as described herein. The autoimmune disease can be Crohn's disease, Irritable Bowel Disease (IBD), multiple sclerosis, type 1 diabetes, psoriasis, systemic lupus erythematosis, or rheumatoid arthritis.

In another aspect, the invention features methods of treating a patient who is suffering from an ischemia-reperfusion injury or anoxia-hypoxia. The methods include a step of administering to the patient a therapeutically effective amount of a pharmaceutical composition as described herein. The ischemia-reperfusion injury or the anoxia-hypoxia can be associated with acute kidney injury or myocardial infarction.

In another aspect, the invention features methods of determining whether a patient is likely to benefit from the administration of a pharmaceutical composition as described herein. The methods can include the step of providing a sample obtained from the patient and determining whether the sample includes an elevated level of RORγt, wherein an elevated level indicates that the patient is likely to benefit from administration of the pharmaceutical composition.

In any of the methods of the invention, or in the context of any use, the patient can be a mammal, and the mammal can be a human.

In another aspect, the invention features use of a composition as described herein in the preparation of a medicament and use in the preparation of a medicament for treating autoimmune disease, graft rejection, or an ischemia reperfusion injury.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is a graph depicting cardiac allograft survival in a C57BL/6 to Balb/c transplant model. The results suggest a synergistic effect with a combined IL-6/IL-21-directed strategy.

FIGS. 2A-2D are graphs representing the ratio of CD4⁺ and CD8⁺ T-cells in the rejecting heart on day 7, WT→WT combination (FIG. 2A); the proportion of Foxp3 (GFP)-expressing cells within the CD4⁺ population in the WT heart on day 7 (FIG. 2B); the CD4/CD8 T-cell ratio in the IL-6 KO heart on day 7 post-transplant, IL-6 KO→WT combination (FIG. 2C); and the proportion of Foxp3 (GFP)-expressing cells within CD4⁺ population in the IL-6 KO heart on day 7 (FIG. 2D).

FIG. 3 is a representation of an amino acid sequence of a human IL-21 antagonist (SEQ ID NO: 1) as disclosed in U.S. Patent Application Publication No. 2006/0039902.

FIG. 4 is a graph depicting the effect of administration of IL-6 on islet allograft rejection and induction of tolerance.

### DETAILED DESCRIPTION

IL-6 is known in the art and has been studied extensively. This cytokine is also known as B-cell stimulatory factor-2 (BSF2) due to its involvement in differentiating B-cells into antibody-producing cells. An early study reported an IL-6 cDNA encoding a protein comprising 184 amino acid residues accompanied by a signal peptide consisting of 28 amino acid residues (Hirano et al., Nature 324:73-76, 1986). The NCBI reference sequence for IL-6 can be found at GENBANK under accession number NP_{—}000591.1 GI:10834984. Despite considerable differences between the human and murine sequences, there seems to be little if any species restriction. IL-6 receptors (IL-6R) are also known in the art. For example, the human IL-6R has been cloned from the natural killer-like cell line YT (Yamasaki et al., Science 241:825, 1988).

IL-6 receptors are expressed in high numbers on certain tumor cell lines such as human myelomas, histiocytomas and promyelocytic leukemia cells (Taga et al., J. Exp. Med. 166:967, 1987). The IL6 receptor is a protein complex consisting of a IL-6 receptor subunit (IL6R) and interleukin 6 signal transducer Glycoprotein 130. Alternatively spliced transcript variants encoding distinct isoforms have been reported. IL6R subunit is also shared by many other cytokines. An exemplary IL-6R alpha subunit for isoform 1 is found at GENBANK under accession number NP_{—}000556.1 GI:4504673.

IL-21 (also known as Za-11) and its receptor (also known as MU-1) are also known in the art, and an exemplary IL-21R cDNA has been deposited with the American Type Culture Collection (Mar. 10, 1998, as accession number ATCC 98687). The nucleotide sequence and amino acid sequence of a human IL-21 is available at GENBANK® under accession number X_{—}011082. Two isoforms of IL-21, based on alternative splice variants, have been described, The NCBI Reference Sequence for isoform 1 can be found at GENBANK under accession number NP_{—}068575.1 (GI:11141875). We refer to the amino acid sequence of GENBANK under accession number NP_{—}068575.1 (GI:11141875) as SEQ ID NO: 2 as shown below:

```
(SEQ ID NO.: 2)
    1 mrsspgnmer iviclmvifl gtlvhksssq gqdrhmirmr qlidivdqlk nyvndlvpef
  61 lpapedvetn cewsafscfq kaqlksantg nneriinvsi kklkrkppst nagrrqkhrl
121 tcpscdsyek kppkeflerf ksllqkmihq hlssrthgse ds.
```

An IL-21 antagonist may have an amino acid sequence that differs from the SEQ ID NO: 2. For example, an antagonist can haveone or more mutations in the 4^{th} helix of IL-21, i.e., at positions 133-152. Exemplary mutations include substitutions at one or both of positions 145 and 150. Useful substitutions include alanine for glutamine.

Isoform two of IL-21 differs from isoform one by having a substitution at amino acids 140-155: MIHQHLSSRTHGSEDS→VSTLSFI. The NCBI Reference Sequence for isoform 2 can be found at Genbank NP_{—}001193935.1 (GI:333033767).

Additional entries providing amino acid sequences for human IL-21 polypeptides are disclosed in U.S. Patent Application Publication No. 2006/0159655 (which is incorporated herein by reference in its entirety). Exemplary entries providing amino acid sequences for human IL-21 polypeptides include: gi|11141875|ref|NP.sub.-068575.1| interleukin 21 [Homo sapiens]; gi|11093536|gb|AAG29348.1| interleukin 21 [Homo sapiens]; gi|42542586|gb|AAH66259.1| Interleukin 21 [Homo sapiens]; gi|42542588|gb|AAH66260.1| Interleukin 21 [Homo sapiens]; gi|42542657|gb|AAH66261.1| Interleukin 21 [Homo sapiens]; gi|42542659|gb|AAH66258.1| Interleukin 21 [Homo sapiens]; and gi|42542807|gb|AAH66262.1| Interleukin 21 [Homo sapiens]. The IL-21 polypeptide can be a variant of a polypeptide described herein, provided that it retains functionality.

The IL-21 polypeptide can be encoded, for example, by NM_{—}021803.2 GI:190886440 or NM_{—}001207006.1 GI:333033766.

The IL-21R is a class I cytokine family receptor, also known as NILR (see WO 01/85792; Parrish-Novak et al., Nature 408:57-63, 2000; and Ozaki et al., Proc. Natl. Acad. Sci. USA 97:11439-11444, 2000). The IL-21R is homologous to the shared β chain of the IL-2 and IL-15 receptors, and the IL-4 receptor a chain (Ozaki et al., supra). Upon ligand binding, the IL-21R is capable of interacting with a common γ cytokine receptor chain (γc) (Asao et al., J. Immunol. 167:1-5, 2001), and inducing the phosphorylation of STAT1 and STAT3 or STAT5. The receptor shows widespread lymphoid tissue distribution.

Exemplary sequences for IL21-R can be found at GENBANK Accession Numbers NP_{—}068570.1 GI:11141869; NP 851564.1 GI:31083174; and NP_{—}851565.4 GI:302034748. Three alternatively spliced transcript variants encoding the same IL-21R polpeptide have been described.

Peptide antagonists useful in the present compositions and methods can be fragments or other mutants of the IL-6 cytokine itself or a fragment or other mutant of the IL-6 receptor molecule. U.S. Pat. No. 5,210,075 (the content of which is hereby incorporated by reference in its entirety) describes peptides having activity as IL-6 antagonists, and these peptides can be used in the context of the present combination therapies and formulations. As described further in the '075 patent, useful peptides can be derived from (e.g., can comprise) the p51-70 portion of IL-6 (see Hirano et al., Nature 324:73, 1986), which has the amino acid sequence ESSKEALAENNLNLPKMAEK (SEQ ID NO: 3). In this fragment or others, one can incorporate additional amino acids (such as a terminal cysteine). Other exemplary peptide antagonists comprising portions of IL-6 include peptides having the amino acid sequence GALAENNLNLP (SEQ ID NO: 4); ESSKEALAENN (SEQ ID NO: 5); NLNLPKMAEK (SEQ ID NO: 6). Exemplary peptide antagonists comprising portions of IL-6R include peptides having the amino acid sequence RHVVQLRAQEEFGQGEWSEWS (SEQ ID NO: 7) derived from the p268-289 portion of the IL-6R; RHVVQLRAQEEF (SEQ ID NO: 8); GQLRAQEEFGQGE (SEQ ID NO: 9); EFGQGEWSEWS (SEQ ID NO: 10); AGSHPSRWAGMGRRLLLR (SEQ ID NO: 11) derived from the p48-66 portion of the IL-6R; HPSRWAGMGRR (SEQ ID NO: 12); AGSHPSRWAG (SEQ ID NO: 13); CGHPSRWAGMGRR (SEQ ID NO: 14); GMGRRLLLRS (SEQ ID NO: 15); MVKDLQHHCVIHDAWSG (SEQ ID NO: 16) derived from the p250-267 portion of the IL-6R; MVKDLQHHCVIHDA (SEQ ID NO: 17); RLFQHSPAGDFQEPCQYSQESQLF (SEQ ID NO: 18); EPSQYSQESQLF (SEQ ID NO: 19); AGDFQEPSQYSQE (SEQ ID NO: 20); RLFQNSPAGDFQEPCQYSQESQLF (SEQ ID NO: 21) derived from the p132-155 region of the IL-6R. Where an IL-6 or IL-6R fragment is incorporated into a fusion protein, the antagonistic peptide can include one or more adjacent internal glycine spacer residues to facilitate coupling. The same is true for IL-21 and IL-21R peptide antagonists.

D-form amino acid residues may be incorporated, as these may protect the peptide antagonist from metabolism in the in vivo environment and thereby increase the effective half-life of the compound in the body. Such substitutions can be employed at the amino- and/or carboxy-terminal of the peptide. IL-6 or IL-6R peptide antagonists can also include substituents such as lower (C₁-C₈) alkoxyl groups and single-ring aroyl groups. The same is true for IL-21 and IL-21R peptide antagonists.

U.S. Pat. No. 5,789,552 (hereby incorporated by reference in its entirety) references a three-dimensional model of IL-6 that enabled the identification of two sites of interaction between human IL-6 and its two receptors: the low affinity receptor gp 80 (site 1) and the high affinity receptor for gp 130 signal transduction (site 2). These receptor antagonists, which are useful in the context of the present combination therapies, are generated by mutating amino acid positions 31, 35, 118, 121, 175, 176 and/or 183 of human IL-6. Peptide antagonists described in U.S. Pat. No. 5,849,283 (hereby incorporated by reference in its entirety) can also be used in the context of the present invention. Exemplary peptide antagonists of the IL-6 R include mutant IL-6 having a substitution of Asp for Tyr31; a substitution of Tyr, Phe or Leu for Gly35; a substitution of Arg, Phe or Leu for Ser118; a substitution of Asp for Val121; a substitution of Ile for Gln 175; a substitution of Arg for Ser 176; a substitution of Ala for Gln 183.

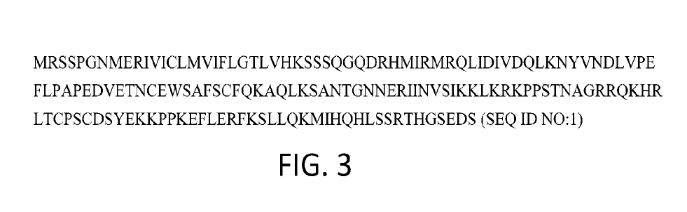

U.S. Pat. No. 7,198,789 and U.S. Patent Application Publication No. 2006/0039902 (the contents of which are hereby incorporated by reference in their entirety) describe peptides having activity as IL-21 antagonists, and these peptides can be used in the context of the present combination therapies and formulations. More specifically, an IL-21 antagonist can have the sequence shown in FIG. 3 or be a fragment or other mutant thereof. The IL-21 antagonist can be an IL-21-binding fragment of a soluble IL-21 receptor. For example, the IL-21 antagonist can be or can include the extracellular domain of the IL-21R. Exemplary fragments include fragments comprising amino acids 1-235, 1-236, 20-235 and 20-236 of the human IL-21R. This domain can be fused to a heterologous protein (e.g., an Fc immunoglobulin region). The fusion protein may be one that ameliorates inflammatory symptoms in collagen-induced arthritis (CIA) animal models or in an animal model of IBD, graft rejection, psoriasis, or lupus.

Peptides that are IL-6 or IL-21 antagonists can be synthesized by the solid phase peptide synthesis (or Merrifield) method, by solution phase synthesis, or by other techniques known in the art. The Merrifield synthesis is well established and widely used (see, e.g., Merrifield, J. Am. Chem. Soc., 85:2149-2154, 1963; Meienhofer, in “Hormonal Proteins and Peptides,” ed. C. H. Li, Vol. 2 (Academic Press, 1973), p. 48-267; and Barany and Merrifield in “The Peptides,” eds. E. Gross and J. Meienhofer, Vol. 2 (Academic Press, 1980), p. 3-285.

Polypeptides: We refer to the amino acid-based compositions of the invention as “polypeptides” to convey that they are linear polymers of amino acid residues, and to help distinguish them from full-length proteins. A polypeptide of the invention can “constitute” or “include” a fragment of an IL-6, an IL-6R, an IL-21 or an IL-21R, and the invention encompasses polypeptides that constitute or include biologically active variants of an IL-6, an IL-6R, an IL-21 or an IL-21R. It will be understood that the polypeptides can therefore include only a fragment of an IL-6, an IL-6R, an IL-21 or an IL-21R (or a biologically active variant thereof) but may include additional residues as well. Biologically active variants will retain sufficient activity to bind to their respective targets. Regardless of the particular amino acid sequence, the biologically active variants of the invention also retain the ability to function as antagonists of their respective targets.

The bonds between the amino acid residues can be conventional peptide bonds or another covalent bond (such as an ester or ether bond), and the polypeptides can be modified by amidation, phosphorylation or glycosylation. A modification can affect the polypeptide backbone and/or one or more side chains. Chemical modifications can be naturally occurring modifications made in vivo following translation of an mRNA encoding the polypeptide (e.g., glycosylation in a bacterial host) or synthetic modifications made in vitro. A biologically active variant of a truncated IL-6, IL-6R, IL-21 or IL-21R can include one or more structural modifications resulting from any combination of naturally occurring (i.e., made naturally in vivo) and synthetic modifications (i.e., naturally occurring or non-naturally occurring modifications made in vitro). Examples of modifications include, but are not limited to, amidation (e.g., replacement of the free carboxyl group at the C-terminus by an amino group); biotinylation (e.g., acylation of lysine or other reactive amino acid residues with a biotin molecule); glycosylation (e.g., addition of a glycosyl group to either asparagines, hydroxylysine, serine or threonine residues to generate a glycoprotein or glycopeptide); acetylation (e.g., the addition of an acetyl group, typically at the N-terminus of a polypeptide); alkylation (e.g., the addition of an alkyl group); isoprenylation (e.g., the addition of an isoprenoid group); lipoylation (e.g. attachment of a lipoate moiety); and phosphorylation (e.g., addition of a phosphate group to serine, tyrosine, threonine or histidine).

One or more of the amino acid residues in a biologically active variant may be a non-naturally occurring amino acid residue. Naturally occurring amino acid residues include those naturally encoded by the genetic code as well as non-standard amino acids (e.g., amino acids having the D-configuration instead of the L-configuration). The present peptides can also include amino acid residues that are modified versions of standard residues (e.g. pyrrolysine can be used in place of lysine and selenocysteine can be used in place of cysteine). Non-naturally occurring amino acid residues are those that have not been found in nature, but that conform to the basic formula of an amino acid and can be incorporated into a peptide. These include D-alloisoleucine(2R,3S)-2-amino-3-methylpentanoic acid and L-cyclopentyl glycine (S)-2-amino-2-cyclopentyl acetic acid. For other examples, one can consult textbooks or the worldwide web (a site is currently maintained by the California Institute of Technology and displays structures of non-natural amino acids that have been successfully incorporated into functional proteins). Non-natural amino acid residues and amino acid derivatives listed in U.S. Application No. 20040204561 (see ¶0042, for example) can also be used.

Alternatively, or in addition, one or more of the amino acid residues in a biologically active variant can be a naturally occurring residue that differs from the naturally occurring residue found in the corresponding position in a wildtype sequence. In other words, biologically active variants can include one or more amino acid substitutions. We may refer to a substitution, addition, or deletion of amino acid residues as a mutation of the wildtype sequence. As noted, the substitution can replace a naturally occurring amino acid residue with a non-naturally occurring residue or just a different naturally occurring residue. Further the substitution can constitute a conservative or non-conservative substitution. Conservative amino acid substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine.

The polypeptides that are biologically active variants of an IL-6, an IL-6R, an IL-21 or an IL-21R polypeptide can be characterized in terms of the extent to which their sequence is similar to or identical to the corresponding wild-type polypeptide. For example, the sequence of a biologically active variant can be at least or about 80% identical to corresponding residues in the wild-type polypeptide. For example, a biologically active variant of an IL-6, an IL-6R, an IL-21 or an IL-21R polypeptide can have an amino acid sequence with at least or about 80% sequence identity (e.g., at least or about 85%, 90%, 95%, 97%, 98%, or 99% sequence identity) to an IL-6, an IL-6R, an IL-21 or an IL-21R polypeptide or to a homolog or ortholog thereof.

A biologically active variant of an IL-6, an IL-6R, an IL-21 or an IL-21R polypeptide will retain sufficient biological activity to be useful in the present methods. The biologically active variants will retain sufficient activity to function as antagonists of their respective targets. The biological activity can be assessed in ways known to one of ordinary skill in the art and includes, without limitation, in vitro binding and competition assays, blockade of signal transduction, or cytotoxicity assays.

Peptides can also, of course, be generated by recombinant techniques.

Once generated, peptides can be isolated and purified to any desired extent by means well known in the art. For example, one can use lyophilization following, for example, reversed phase (preferably) or normal phase HPLC, or size exclusion or partition chromatography on polysaccharide gel media such as Sephadex G-25. The composition of the final peptide may be confirmed by amino acid analysis after degradation of the peptide by standard means, by amino acid sequencing, or by FAB-MS techniques.

Salts, including acid salts, esters, amides, and N-acyl derivatives of an amino group of a peptide antagonist may be prepared using methods known in the art, and such peptides are useful in the context of the present invention.

The sequence of the IL-6R is known in the art and soluble fragments or other mutants of the receptor that bind IL-6, effectively inhibiting its activity, can be used in the compositions and methods of the present invention. The receptor and receptor fragments or other mutants can be as described, for example, in U.S. Pat. No. 5,480,796, which is hereby incorporated by reference herein in its entirety. This patent describes the IL-6R and fragments or other mutants thereof capable of specifically binding to IL-6. The mutants include IL-6R polypeptides wherein one or more amino acid residues is replaced by a different amino acid residue; one or more amino acid residues is deleted; or one or more amino acid residues is added. Specific receptor antagonists are described, and these can be incorporated in the context of the present invention (i.e., used with IL-21 antagonists). As indicated above, the peptide antagonists, including those related to the IL-6 receptor, may be fusion proteins wherein any one of the above-mentioned proteins is fused with another protein or a fragment thereof.

Antibodies: The IL-6 or IL-21 antagonist can be an antibody that specifically binds IL-6, the IL-6 receptor, IL-21, or the IL-21 receptor. We use the term antibody to broadly refer to immunoglobulin-based binding molecules, and the term encompasses conventional antibodies (e.g., the tetrameric antibodies of the G class (e.g., an IgG1)), fragments thereof that retain the ability to bind their intended target (e.g., an Fab′ fragment), and single chain antibodies (scFvs). The antibody may be polyclonal or monoclonal and may be produced by human, mouse, rabbit, sheep or goat cells, or by hybridomas derived from these cells. The antibody can be humanized, chimeric, or bi-specific (i.e., capable of specifically binding IL-6 and IL-21 and/or an IL-6 or IL-21 receptor (e.g., IL-6 and IL-21; IL-6 and IL-21R; or IL-21 and IL-6R) under physiological conditions). Such antibodies are known in the art, some are commercially or publicly available, and others can be readily generated using conventional techniques.

For example, tocilizumab (Actemra®) is a monoclonal antibody that specifically binds the IL-6 receptor (see U.S. Pat. Nos. 5,480,796 and 5,670,373, the entire contents of which are incorporated herein by reference in their entirety). Actemra® can be incorporated in the present compositions and methods as the IL-6 antagonist. The IL-6 antagonist can be an antibody that specifically binds the sequence KTSMHPPYSLGQLVP (SEQ ID NO: 22) of the human IL-6R, such as the antibody produced by hybridoma MT18 (FERM BP-2999) or the antibody produced by hybridoma PM1 (FERM BP-2998). The neutralizing anti-IL-6 antibody, clone MP5-20F3 (BioXCell, New Lebanon, N.H.) can also be used.

Antibodies to the IL-21 receptor are described in U.S. Patent Application Publications 2006/0039902 and 2006/0159655, the entire contents of which are incorporated herein by reference in their entirety).

Regardless of their target, i.e., IL-6, IL-21, IL-6R or IL-21R, the antibodies can assume various configurations and encompass proteins consisting of one or more polypeptides substantially encoded by immunoglobulin genes. Any one of a variety of antibody structures can be used, including the intact antibody, antibody multimers, or antibody fragments or other variants thereof that include functional, antigen-binding regions of the antibody. We may use the term “immunoglobulin” synonymously with “antibody.” The antibodies may be monoclonal or polyclonal in origin. Regardless of the source of the antibody, suitable antibodies include intact antibodies, for example, IgG tetramers having two heavy (H) chains and two light (L) chains, single chain antibodies, chimeric antibodies, humanized antibodies, complementary determining region (CDR)-grafted antibodies as well as antibody fragments, e.g., Fab, Fab′, F(ab′)2, scFv, Fv, and recombinant antibodies derived from such fragments, e.g., camelbodies, microantibodies, diabodies and bispecific antibodies.

An intact antibody is one that comprises an antigen-binding variable region (V_{H} and V_{L}) as well as a light chain constant domain (C_{L}) and heavy chain constant domains, C_{HI}, C_{H2} and C_{H3}. The constant domains may be native sequence constant domains (e.g. human native sequence constant domains) or amino acid sequence variants thereof. As is well known in the art, the V_{H} and V_{L} regions are further subdivided into regions of hypervariability, termed “complementarity determining regions” (CDRs), interspersed with the more conserved framework regions (FRs). The extent of the FRs and CDRs has been defined (see, Kabat et al. Sequences of Proteins of Immunological Interest, Fifth Edition, U.S. Department of Health and Human Services, NIH Publication No. 91-3242, 1991, and Chothia, et al., J. Mol. Biol. 196:901-917 (1987). The CDR of an antibody typically includes amino acid sequences that together define the binding affinity and specificity of the natural Fv region of a native immunoglobulin binding site.

An anti-IL-6, IL-21, IL-6R or IL-21R antibody can be from any class of immunoglobulin, for example, IgA, IgG, IgE, IgD, IgM (as well as subtypes thereof (e.g., IgG₁, IgG₂, IgG₃, and IgG₄)), and the light chains of the immunoglobulin may be of types kappa or lambda. The recognized human immunoglobulin genes include the kappa, lambda, alpha (IgA₁ and IgA₂), gamma (IgG₁, IgG₂, IgG₃, IgG₄), delta, epsilon, and mu constant region genes, as well as the myriad immunoglobulin variable region genes.

The term “antigen-binding portion” of an immunoglobulin or antibody refers generally to a portion of an immunoglobulin that specifically binds to a target, in this case, an epitope comprising amino acid residues on IL-6, IL-21, IL-6R or IL-21R. An antigen-binding portion of an immunoglobulin is therefore a molecule in which one or more immunoglobulin chains are not full length, but which specifically binds to a cellular target. Examples of antigen-binding portions or fragments include: (i) an Fab fragment, a monovalent fragment consisting of the VLC, VHC, CL and CH1 domains; (ii) a F(ab′)₂ fragment, a bivalent fragment comprising two Fab fragments linked by a disulfide bridge at the hinge region; (iii) a Fv fragment consisting of the VLC and VHC domains of a single arm of an antibody, and (v) an isolated CDR having sufficient framework to specifically bind, e.g., an antigen binding portion of a variable region. An antigen-binding portion of a light chain variable region and an antigen binding portion of a heavy chain variable region, e.g., the two domains of the Fv fragment, VLC and VHC, can be joined, using recombinant methods, by a synthetic linker that enables them to be made as a single protein chain in which the VLC and VHC regions pair to form monovalent molecules (known as single chain Fv (scFv); see e.g., Bird et al., Science 242:423-426 (1988); and Huston et al., Proc. Natl. Acad. Sci. USA 85:5879-5883 (1988)). Such scFvs can be a target agent of the present invention and are encompassed by the term “antigen-binding portion” of an antibody.

An “Fv” fragment is the minimum antibody fragment that contains a complete antigen-recognition and binding site. This region consists of a dimer of one heavy chain and one light chain variable domain in tight, con-covalent association. It is in this configuration that three hypervariable regions of each variable domain interact to define an antigen-binding site on the surface of the V_{H}-V_{L} dimer. While six hypervariable regions confer antigen-binding specificity, even a single variable domain (or half of an Fv comprising only three hypervariable regions specific for an antigen) has the ability to recognize and bind antigen, although at a lower affinity than the entire binding site. To improve stability, the VH-VL domains may be connected by a flexible peptide linker such as (Gly₄Ser)₃ to form a single chain Fv or scFV antibody fragment or may be engineered to form a disulfide bond by introducing two cysteine residues in the framework regions to yield a disulfide stabilized Fv (dsFv).

As noted, other useful antibody formats include diabodies, minibodies and bispecific antibodies. A diabody is a homodimer of scFvs that are covalently linked by a short peptide linker (about 5 amino acids or less). By using a linker that is too short to allow pairing between two domains on the same chain, the domains can be forced to pair with the complementary domains of another chain and create two antigen-binding sites (see, e.g., EP 404,097 and WO 93/11161 for additional information regarding diabodies). A diabody variant, (dsFv)₂ or a linear antibody useful in the present compositions and methods includes a pair of tandem Fd segments (V_{H}-C_{H}1-V_{H}-C_{H}1) that form a pair of antigen binding regions (see, e.g., Zapata et al., Prot. Eng. 8:1057 (1995)). Useful minibodies are homodimers of scFv-C_{H3} fusion proteins. In the minibody variant, the Flex minibody, the scFv is fused to the hinge region of IgG1, which is in turn, linked to the CH₃ region by a 10-amino acid linker.

A bispecific antibody, which recognizes two different epitopes, can also be used. A variety of different bispecific antibody formats have been developed. For example, useful bispecific antibodies can be quadromas, i.e., an intact antibody in which each H-L pair is derived from a different antibody. Typically, quadromas are produced by fusion of two different B cell hybridomas, followed by screening of the fused calls to select those that have maintained the expression of both sets of clonotype immunoglobulin genes. Alternatively, a bispecific antibody can be a recombinant antibody. Exemplary formats for bispecific antibodies include, but are not limited to tandem scFvs in which two single chains of different specificity are connected via a peptide linker; diabodies and single chain diabodies. Useful bispecific antibodies can include, for example, bispecific antibodies that bind IL-6 and IL-21, or IL-6R and IL-21R, or IL-6 and IL-21R, or IL-6R and IL21.

Fragments of antibodies are suitable for use in the methods provided so long as they retain the desired specificity of the full-length antibody and/or sufficient specificity to function as IL-6 or IL-21 antagonists. Thus, a fragment of an anti-IL-6, IL-21, IL-6R or IL-21R antibody, as described herein, can retain the ability of the intact antibody to bind to the particular cytokine or cytokine receptor. These antibody portions can be obtained using conventional techniques known to one of ordinary skill in the art, and the portions can be screened for utility in the same manner as intact antibodies are screened as IL-6 or IL-21 antagonists.

Methods for preparing antibody fragments are well known in the art and encompass both biochemical methods (e.g. proteolytic digestion of intact antibodies which may be followed by chemical cross-linking) and recombinant DNA-based methods in which immunoglobulin sequences are genetically engineered to direct the synthesis of the desired fragments. Exemplary biochemical methods are described in U.S. Pat. Nos. 5,855,866; 5,877,289; 5,965,132; 6,093,399; 6,261,535; and 6,004,555. Nucleic acids encoding a chimeric or humanized chain can be expressed to produce a contiguous polypeptide. See, e.g., Cabilly et al., U.S. Pat. No. 4,816,567; Cabilly et al., European Patent No. 0,125,023 B1; Boss et al., U.S. Pat. No. 4,816,397; Boss et al., European Patent No. 0,120,694 B1; Neuberger et al., WO 86/01533; Neuberger et al., European Patent No. 0,194,276 B1; Winter, U.S. Pat. No. 5,225,539; and Winter, European Patent No. 0,239,400 B 1. See also, Newman et al., BioTechnology 10:1455-1460 (1992), regarding CDR-grafted antibodies and Ladner et al. (U.S. Pat. No. 4,946,778) and Bird et al., Science 242:423-426 (1988)) regarding single chain antibodies.

Antibody fragments can be obtained by proteolysis of the whole immunoglobulin by the non-specific thiolprotease, papain. Papain digestion yields two identical antigen-binding fragments, termed “Fab fragments,” each with a single antigen-binding site, and a residual “Fc fragment.” The various fractions can be separated by protein A-Sepharose or ion exchange chromatography. The usual procedure for preparation of F(ab′)₂ fragments from IgG of rabbit and human origin is limited proteolysis by the enzyme pepsin. Pepsin treatment of intact antibodies yields an F(ab′)₂ fragment that has two antigen-combining sites and is still capable of cross-linking antigen. A Fab fragment contains the constant domain of the light chain and the first constant domain (CH1) of the heavy chain. Fab′ fragments differ from Fab fragments by the addition of a few residues at the carboxyl terminus of the heavy chain CH1 domain including one or more cysteine(s) from the antibody hinge region. F(ab′)₂ antibody fragments were originally produced as pairs of Fab′ fragments that have hinge cysteines between them. Other chemical couplings of antibody fragments are known.

Also within the scope of the present invention are methods of making a targeting agent (e.g., an antibody or an antigen-binding fragment or other variant thereof) that targets IL-6, IL-21, IL-6R or IL-21R (or to an epitope including amino acid residues in two or more of these subdomains). For example, variable regions can be constructed using PCR mutagenesis methods to alter DNA sequences encoding an immunoglobulin chain (e.g., using methods employed to generate humanized immunoglobulins; see e.g., Kanunan et al., Nucl. Acids Res. 17:5404 (1989); Sato et al., Cancer Research 53:851-856 (1993); Daugherty et al., Nucleic Acids Res. 19(9):2471-2476 (1991); and Lewis and Crowe, Gene 101:297-302 (1991)). Using these or other suitable methods, variants can also be readily produced. For example, in one embodiment, cloned variable regions can be mutagenized, and sequences encoding variants with the desired specificity can be selected (e.g., from a phage library; see e.g., Krebber et al., U.S. Pat. No. 5,514,548; and Hoogenboom et al., WO 93/06213).

Other suitable methods of producing or isolating immunoglobulins that specifically recognize a cellular target as described herein include, for example, methods that rely upon immunization of transgenic animals (e.g., mice) capable of producing a full repertoire of human antibodies (see e.g., Jakobovits et al., Proc. Natl. Acad. Sci. USA 90:2551-2555 (1993); Jakobovits et al., Nature 362:255-258 (1993); Lonberg et al., U.S. Pat. No. 5,545,806; and Surani et al., U.S. Pat. No. 5,545,807).

As is well known in the art, monoclonal antibodies are homogeneous antibodies of identical antigenic specificity produced by a single clone of antibody-producing cells, and polyclonal antibodies generally recognize different epitopes on the same antigen and are produced by more than one clone of antibody producing cells. Each monoclonal antibody is directed against a single determinant on the antigen. The modifier, monoclonal, indicates the character of the antibody as being obtained from a substantially homogeneous population of antibodies, and is not to be construed as requiring production of the antibody by any particular method. For example, the monoclonal antibodies may be made by the hybridoma method first described by Kohler et al., (Nature 256:495 (1975)) or by recombinant DNA methods (see, e.g., U.S. Pat. No. 4,816,567). The monoclonal antibodies may also be isolated from phage antibody libraries using the techniques described in Clackson et al. (Nature 352:624-628 (1991)) and Marks et al., (J. Mol. Biol. 222:581-597 (1991)), for example.

The monoclonal antibodies herein can include chimeric antibodies, i.e., antibodies that typically have a portion of the heavy and/or light chain identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)). Chimeric antibodies of interest include primatized antibodies comprising variable domain antigen-binding sequences derived from a non-human primate (e.g. apes, Old World monkeys, New World monkeys, prosimians) and human constant region sequences.

Methods for producing monoclonal antibodies can include purification steps. For example, the antibodies can generally can be further purified, for example, using filtration, centrifugation and various chromatographic methods, such as HPLC or affinity chromatography, all of which are techniques well known to one of ordinary skill in the art. These purification techniques each involve fractionation to separate the desired antibody from other components of a mixture. Analytical methods particularly suited to the preparation of antibodies include, for example, protein A-Sepharose and/or protein G-Sepharose chromatography.

The anti-IL-6, IL-21, IL-6R and IL-21R antibodies of the invention may include CDRs from a human or non-human source. “Humanized” antibodies are generally chimeric or mutant monoclonal antibodies from mouse, rat, hamster, rabbit or other species, bearing human constant and/or variable region domains or specific changes. Techniques for generating a so-called “humanized” antibody are well known to those of skill in the art.

The framework of the immunoglobulin can be human, humanized, or non-human (e.g., a murine framework modified to decrease antigenicity in humans), or a synthetic framework (e.g., a consensus sequence). Humanized immunoglobulins are those in which the framework residues correspond to human germline sequences and the CDRs result from V(D)J recombination and somatic mutations. However, humanized immunoglobulins may also comprise amino acid residues not encoded in human germline immunoglobulin nucleic acid sequences (e.g., mutations introduced by random or site-specific mutagenesis ex vivo). It has been demonstrated that in vivo somatic mutation of human variable genes results in mutation of framework residues (see Nature Immunol. 2:537 (2001)). Such an antibody would be termed “human” given its source, despite the framework mutations. Mouse antibody variable domains also contain somatic mutations in framework residues (See Sem. Immunol. 8:159 (1996)). Consequently, transgenic mice containing the human Ig locus produce immunoglobulins that are commonly referred to as “fully human,” even though they possess an average of 4.5 framework mutations (Nature Genet. 15:146-56 (1997)). Accepted usage therefore indicates that an antibody variable domain gene based on germline sequence but possessing framework mutations introduced by, for example, an in vivo somatic mutational process is termed “human.”

Humanized antibodies may be engineered by a variety of methods known in the art including, for example: (1) grafting the non-human complementarity determining regions (CDRs) onto a human framework and constant region (a process referred to in the art as humanizing), or, alternatively, (2) transplanting the entire non-human variable domains, but providing them with a human-like surface by replacement of surface residues (a process referred to in the art as veneering). Humanized antibodies can include both humanized and veneered antibodies. Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545.806; 5,569,825; 5,625,126; 5,633,425; 5,661,016, and in the following scientific publications: Marks et al., Bio/Technology 10:779-783 (1992); Lonberg et al. Nature 368:856-859 (1994); Morrison, Nature 368:812-13 (1994); Fishwild et al., Nature Biotechnology 14:845-51 (1996): Neuberger, Nature Biotechnology 14:826 (1996); Lonberg and Huszar, Intern. Rev. Immunol. 13:65-93 (1995); Jones et al., Nature 321:522-525 (1986); Morrison et al., Proc. Acad. Sci. USA, 81:6851-6855 (1984); Morrison and Oi, Adv. Immunol. 44:65-92 (1988); Verhoeyer et al., Science 239:1534-1536 (1988); Padlan, Molec. Immun. 28:489-498 (1991); Padlan, Molec. Immunol. 31(3):169-217 (1994); and Kettleborough, C. A. et al., Protein Eng. 4(7):773-83 (1991)).

In addition to chimeric and humanized antibodies, fully human antibodies can be derived from transgenic mice having human immunoglobulin genes (see, e.g., U.S. Pat. Nos. 6,075,181; 6,091,001; and 6,114,598), or from phage display libraries of human immunoglobulin genes (see, e.g. McCafferty et al., Nature 348:552-554 (1990); Clackson et al., Nature 352:624-628 (1991), and Marks et al., J. Mol. Biol. 222:581-597 (1991)). In some embodiments, antibodies may be produced and identified by scFv-phage display libraries using standard methods known in the art.

The anti-IL-6, IL-21, IL-6R and IL-21R antibodies may be modified to modulate their antigen binding affinity, their effector functions, or their pharmacokinetics. In particular, random mutations can be made in the CDRs and products screened to identity antibodies with higher affinities and/or higher specificities. Such mutagenesis and selection is routinely practiced in the antibody arts. A convenient way for generating such substitutional variants is affinity maturation using phage display.

CDR shuffling and implantation technologies can be used with the antibodies provided herein, for example. CDR shuffling inserts CDR sequences into a specific framework region (Jirholt et al., Gene 215:471 (1988)). CDR implantation techniques permit random combination of CDR sequences into a single master framework (Soderlind et al., Immunotechnol. 4:279 (1999); and Soderlind et al., Nature Biotechnol. 18:852 (2000)). Using such techniques, CDR sequences of the anti-IL-6, IL-21, IL-6R or IL-21R antibody, for example, can be mutagenized to create a plurality of different sequences, which can be incorporated into a scaffold sequence and the resultant antibody variants screened for desired characteristics, e.g., higher affinity. In some embodiments, sequences of the anti-anti-IL-6, IL-21, IL-6R and IL-21 antibody can be examined for the presence of T cell epitopes, as is known in the art. The underlying sequence can then be changed to remove T cell epitopes, i.e., to “deimmunize” the antibody.

Recombinant technology using, for example phagemid technology, allows for preparation of antibodies having a desired specificity from recombinant genes encoding a range of antibodies. Certain recombinant techniques involve isolation of antibody genes by immunological screening of combinatorial immunoglobulin phage expression libraries prepared from RNA isolated from spleen of an immunized animal (Morrison et al., Mt. Sinai J. Med. 53:175 (1986); Winter and Milstein, Nature 349:293 (1991); Barbas et al., Proc. Natl. Acad. Sci. USA 89:4457 (1992)). For such methods, combinatorial immunoglobulin phagemid libraries can be prepared from RNA isolated from spleen of an immunized animal, and phagemids expressing appropriate antibodies can be selected by panning using cells expressing antigen and control cells. Advantage of this approach over conventional hybridoma techniques include approximately 10⁴ times as many antibodies can be produced and screened in a single round, and that new specificities can be generated by H and L chain combination, which can further increase the percentage of appropriate antibodies generated. Methods of making phagemid libraries are well known in the art.

In addition to the combinatorial immunoglobulin phage expression libraries disclosed above, one molecular cloning approach is to prepare antibodies from transgenic mice containing human antibody libraries. Such techniques are described (U.S. Pat. No. 5,545,807, incorporated herein by reference). Such transgenic animals can be employed to produce human antibodies of a single isotype, more specifically an isotype that is essential for 13 cell maturation, such as IgM and possibly IgD. Another method for producing human antibodies is described in U.S. Pat. Nos. 5,545,806; 5,569,825; 5,625,126; 5,633,425; 5,661,016; and 5,770,429, wherein transgenic animals are described that are capable of switching from an isotype needed for B cell development to other isotypes.

The anti-IL-6, IL-21, IL-6R and IL-21R immunoglobulins may be modified to reduce or abolish glycosylation. An immunoglobulin that lacks glycosylation may be an immunoglobulin that is not glycosylated at all; that is not fully glycosylated; or that is atypically glycosylated (i.e., the glycosylation pattern for the mutant differs from the glycosylation pattern of the corresponding wild type immunoglobulin). The IgG polypeptides include one or more (e.g., 1, 2, or 3 or more) mutations that attenuate glycosylation, i.e., mutations that result in an IgG CH2 domain that lacks glycosylation, or is not fully glycosylated or is atypicially glycosylated. Mutations of the asparagine residue at amino acid 297 in human IgG 1 is an example of such a mutation. The oligosaccharide structure can also be modified, for example, by eliminating the fusose moiety from the N-linked glycan.

Antibodies can also be modified to increase their stability and or solubility in vivo by conjugation to non-protein polymers, e.g, polyethylene glycol. Any PEGylation method can be used as long as the anti-Il-6, IL-21, IL-6R or IL-21R antibody retains the ability to selectively bind the respective target.

A wide variety of antibody/immunoglobulin frameworks or scaffolds can be employed so long as the resulting polypeptide includes at least one binding region that is specific for the target, i.e., IL-6, IL-21, IL-6R or IL-21R. Such frameworks or scaffolds include the five main idiotypes of human immunoglobulins, or fragments thereof (such as those disclosed elsewhere herein), and include immunoglobulins of other animal species, preferably having humanized aspects. Single heavy-chain antibodies such as those identified in camelids are of particular interest in this regard.

One can generate non-immunoglobulin based antibodies using non-immunoglobulin scaffolds onto which CDRs of the anti-IL-6, IL-21, IL-6R or IL-21R antibody can be grafted. Any non-immunoglobulin framework and scaffold know to those in the art may be used, as long as the framework or scaffold includes a binding region specific for the target. Immunoglobulin-like molecules include proteins that share certain structural features with immunoglobulins, for example, a β-sheet secondary structure. Examples of non-immunoglobulin frameworks or scaffolds include, but are not limited to, adnectins (fibronectin), ankyrin, domain antibodies and Ablynx nv, lipocalin, small modular immuno-pharmaceuticals (Trubion Pharmaceuticals Inc., Seattle, Wash.), maxybodies (Avidia, Inc., Mountain View, Calif.), Protein A and affilin (gamma-crystallin or ubiquitin) (Scil Proteins GmbH, Halle, Germany).

The anti-IL-6, IL-21. IL-6R and IL-21R antibodies of the invention specifically bind to an epitope on IL-6, IL-21, IL6R and IL-21R, respectively. An epitope refers to an antigenic determinant on a target that is specifically bound by the paratope, i.e., the binding site of an antibody. Epitopic determinants usually consist of chemically active surface groupings of molecules such as amino acids or sugar side chains, and typically have specific three-dimensional structural characteristics, as well as specific charge characteristics. Epitopes generally have between about 4 to about 10 contiguous amino acids (a continuous epitope), or alternatively can be a set of noncontiguous amino acids that define a particular structure (e.g., a conformational epitope). Thus, an epitope can consist of at least 4, at least 6, at least 8, at least 10, and at least 12 such amino acids. Methods of determining the spatial conformation of amino acids are known in the art, and include, for example, x-ray crystallography and 2-dimensional nuclear magnetic resonance.

Methods of predicting other potential epitopes to which an antibody can bind are well-known to those of skill in the art and include without limitation, Kyte-Doolittle Analysis (Kyte and Dolittle, J. Mol. Biol. 157:105-132 (1982)), Hopp and Woods Analysis (Hopp and Woods, Proc. Natl. Acad. Sci. USA 78:3824-3828 (1981); Hopp and Woods, Mol. Immunol. 20:483-489 (1983); Hopp, J. Immunol. Methods 88:1-18 (1986)), Jameson-Wolf Analysis (Jameson and Wolf, Comput. Appl. Biosci. 4:181-186 (1988)), and Emini Analysis (Emini et al., Virology 140:13-20 (1985)). In some embodiments, potential epitopes are identified by determining theoretical extracellular domains. Analysis algorithms such as TMpred (see Hofmann and Stoffel, Biol. Chem. 374:166 (1993)) or TMHMM (Krogh et al., J. Mol. Biol. 305(3):567-580 (2001)) can be used to make such predictions. Other algorithms, such as SignalP 3.0 (Bednsten et al., J. Mol. Biol. 340(4):783-795 (2004)) can be used to predict the presence of signal peptides and to predict where those peptides would be cleaved from the full-length protein. The portions of the proteins on the outside of the cell can serve as targets for antibody interaction.

The compositions of the present invention include antibodies that (1) exhibit a threshold level of binding activity; and/or (2) do not significantly cross-react with known related polypeptide molecules. The binding affinity of an antibody can be readily determined by one of ordinary skill in the art, for example, by Scatchard analysis (Scatchard, Ann. NY Acad. Sci. 51:660-672 (1949)).

In some embodiments, the anti-IL-6, IL-21, IL-6R and IL-21R antibodies can bind to their target epitopes or mimetic decoys at least 1.5-fold, 2-fold, 5-fold 10-fold, 100-fold, 10³-fold, 10⁴-fold, 10⁵-fold, 10⁶-fold or greater for the target anti-IL-6, IL-21, IL-6R and IL-21R than to other proteins predicted to have some homology to IL-6, IL-21, IL-6R and IL-21R.

In some embodiments the anti-IL-6, IL-21, IL-6R and IL-21R antibodies bind with high affinity of 10⁻⁴M or less, 10⁻⁷M or less, 10⁻⁹ M or less or with subnanomolar affinity (0.9, 0.8, 0.7, 0.6, 0.5, 0.4, 0.3, 0.2, 0.1 nM or even less). In some embodiments the binding affinity of the anti-IL-6, IL-21, IL-6R and IL-21R antibodies for their respective targets is at least 1×10⁶ Ka. In some embodiments the binding affinity of the anti-IL-6, IL-21, IL-6R and IL-21R antibodies for IL-6, IL-21, IL-6R and IL-21R, respectively, is at least 5×10⁶ Ka, at least 1×10⁷ Ka, at least 2×10⁷ Ka, at least 1×10⁸ Ka, or greater. Antibodies may also be described or specified in terms of their binding affinity to IL-6, IL-21, IL-6R and IL-21R. In some embodiments binding affinities include those with a Kd less than 5×10⁻² M, 10⁻² M, 5×10⁻³ M, 10⁻³ M, 5×10⁻³ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸M, 5×10⁻⁹ M, 5×10⁻¹⁰ M, 10⁻¹⁰ M, 5×10⁻¹¹ M, 10⁻¹¹ M, 5×10⁻¹²M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M, or less.

In some embodiments, the antibodies do not bind to known related polypeptide molecules; for example, they bind IL-6, IL-21, IL-6R and IL-21R but not known related polypeptides. Antibodies may be screened against known related polypeptides to isolate an antibody population that specifically binds IL-6, IL-21, IL-6R and IL-21R. For example, antibodies specific to IL-6, IL-21, IL-6R and IL-21R will flow through a column comprising IL-6, IL-21, IL-6R and IL-21R (with the exception of IL-6, IL-21, IL-6R and IL-21R) adhered to insoluble matrix under appropriate buffer conditions. Such screening allows isolation of polyclonal and monoclonal antibodies non-crossreactive to closely related polypeptides (Antibodies: A Laboratory Manual, Harlow and Lane (eds.), Cold Spring Harbor Laboratory Press, 1988; Current so Protocols in Immunology, Cooligan et al. (eds.), National Institutes of Health, John Wiley and Sons, Inc., 1995). Screening and isolation of specific antibodies is well known in the art (see. Fundamental Immunology. Paul (eds.), Raven Press, 1993; Getzoff et al., Adv. in Immunol. 43:1-98 (1988); Monoclonal Antibodies: Principles and Practice, Goding, J. W. (eds.), Academic Press Ltd., 1996; Benjamin et al., Ann. Rev. Immunol. 2:67-101, 1984). Representative examples of such assays include: concurrent immunoelectrophoresis, radioimmunoassay (RIA), radioimmunoprecipitation, enzyme-linked immunosorbent assay (ELISA), dot blot or Western blot assay, inhibition or competition assay, and sandwich assay.

The ability of a particular antibody to selectively kill IL-6, IL-21, IL-6R and IL-21R expressing cells can be evaluated using standard methods known in the art.

The anti-IL-6. IL-21. IL-6R and IL-21R antibodies can include a tag, which may also be referred to as a reporter or marker (e.g., a detectable marker). A detectable marker can be any molecule that is covalently linked to the anti-IL-6, IL-21, IL-6R and IL-21R antibody or a biologically active fragment thereof that allows for qualitative and/or quantitative assessment of the expression or activity of the tagged peptide. The activity can include a biological activity, a physico-chemical activity, or a combination thereof. Both the form and position of the detectable marker can vary, as long as the labeled antibody retains biological activity. Many different markers can be used, and the choice of a particular marker will depend upon the desired application. Labeled anti-IL-6, IL-21, IL-6R and IL-21R antibodies can be used, for example, for assessing the levels of IL-6, IL-21, IL-6R and IL-21R in a biological sample, e.g., urine, saliva, cerebrospinal fluid, blood or a biopsy sample or for evaluation the clinical response to IL-6, IL-21, IL-6R and IL-21R peptide therapeutics.

Exemplary detectable labels include a radiopaque or contrast agents such as barium, diatrizoate, ethiodized oil, gallium citrate, iocarmic acid, iocetamic acid, iodamide, iodipamide, iodoxamic acid, iogulamide, iohexyl, iopamidol, iopanoic acid, ioprocemic acid, iosefamic acid, ioseric acid, iosulamide meglumine, iosemetic acid, iotasul, iotetric acid, iothalamic acid, iotroxic acid, ioxaglic acid, ioxotrizoic acid, ipodate, meglumine, metrizamide, metrizoate, propyliodone, and thallous chloride. Alternatively or in addition, the detectable label can be a fluorescent label, for example, fluorescein isothiocyanate, rhodamine, phycoerytherin, phycocyanin, allophycocyanin, o-phthaldehyde and fluorescamine; a chemiluminescent compound selected from the group consisting of luminol, isoluminol, an aromatic acridinium ester, an imidazole, an acridinium salt and an oxalate ester; a liposome or dextran; or a bioluminescent compound such as luciferin, luciferase and aequorin.

Suitable markers include, for example, enzymes, photo-affinity ligands, radioisotopes, and fluorescent or chemiluminescent compounds. Methods of introducing detectable markers into peptides are well known in the art. Markers can be added during synthesis or post-synthetically. Recombinant IL-6, IL-21. IL-6R and IL-21R antibodies or biologically active variants thereof can also be labeled by the addition of labeled precursors (e.g. radiolabeled amino acids) to the culture medium in which the transformed cells are grown. In some embodiments, analogues or variants of peptides can be used in order to facilitate incorporation of detectable markers. For example, any N-terminal phenylalanine residue can be replaced with a closely related aromatic amino acid, such as tyrosine, that can be easily labeled with ¹²⁵I. In some embodiments, additional functional groups that support effective labeling can be added to the fragments of an anti-IL-6, IL-21, IL-6R and IL-21R antibody or biologically active variants thereof. For example, a 3-tributyltinbenzoyl group can be added to the N-terminus of the native structure; subsequent displacement of the tributyltin group with ¹²⁵I will generate a radiolabeled iodobenzoyl group.

Any art-known method can be used for detecting such labels, for example, positron-emission tomography (PET), SPECT imaging, magnetic resonance imaging, X-ray; or is detectable by ultrasound.

Oligonucleotides and microRNAs can also be used as antagonists of IL-6 and IL-21 as they can effectively downregulate expression of either the cytokines per se or their receptors.

Pharmaceutical Formulations:

The IL-6 antagonist and the IL-21 antagonist can be administered directly to a mammal. Generally, the antagonists can be suspended in a pharmaceutically acceptable carrier (e.g., physiological saline or a buffered saline solution) to facilitate their delivery. Encapsulation of the antagonists in a suitable delivery vehicle (e.g., polymeric microparticles or implantable devices) may increase the efficiency of delivery. A composition can be made by combining any of the antagonists provided herein with a pharmaceutically acceptable carrier. Where the IL-6 antagonist and the IL-21 antagonist are administered to the same patient, they can be administered in a single formulation or in separate formulations (which may be the same or different) that are administered concurrently or sequentially.

The antagonists can be formulated in various ways for parenteral or non-parenteral administration. Where the antagonist is applied to an organ, tissue, or cells prior to transplantation, the antagonist can be applied directly, such as by means of a solution, suspension, or infusion. Where the antagonist is administered systemically, it can be administered intravenously or by other non-parenteral routes. Where suitable, oral formulations can take the form of tablets, pills, capsules, or powders, which may be enterically coated or otherwise protected. Sustained release formulations, suspensions, elixirs, aerosols, and the like can also be used.

Pharmaceutically acceptable carriers and excipients can be incorporated (e.g., water, saline, aqueous dextrose, and glycols, oils (including those of petroleum, animal, vegetable or synthetic origin), starch, cellulose, talc, glucose, lactose, sucrose, gelatin, malt, rice, flour, chalk, silica gel, magnesium stearate, sodium stearate, glycerol monosterate, sodium chloride, dried skim milk, glycerol, propylene glycol, ethanol, and the like). The compositions may be subjected to conventional pharmaceutical expedients such as sterilization and may contain conventional pharmaceutical additives such as preservatives, stabilizing agents, wetting or emulsifying agents, salts for adjusting osmotic pressure, buffers, and the like. Suitable pharmaceutical carriers and their formulations are described in “Remington's Pharmaceutical Sciences” by E. W. Martin. Such compositions will, in any event, contain an effective amount of the antagonist(s) together with a suitable amount of carrier so as to prepare the proper dosage form for proper administration to the patient or to a graft-donor.

The methods of the present invention can be instigated when a patient is specifically in need of immediate relief from the symptoms of graft rejection or autoimmune disease or to preclude or lessen the risk of or severity of such symptoms. Thus, the present methods can include a step of identifying a patient in need of treatment.

Any composition described herein can be administered to any part of the host's body for subsequent delivery to an IL-6 or IL-21 responsive cell. Alternatively or in addition, the compositions can be administered to target circulating IL-6 or IL-21. A composition can be delivered to, without limitation, the bones, bone marrow, joints, nasal mucosa, blood, lungs, intestines, muscle tissues, skin, or the peritoneal cavity of a mammal. In terms of routes of delivery, a composition can be administered by intravenous, intraperitoneal, intramuscular, subcutaneous, intramuscular, intrarectal, intravaginal, intrathecal, intratracheal, intradermal, or transdermal injection, by oral or nasal administration, or by gradual perfusion over time. In a further example, an aerosol preparation of a composition can be given to a host by inhalation.

The dosage required will depend on the route of administration, the nature of the formulation, the nature of the patient's illness, the patient's size, weight, surface area, age, and sex, other drugs being administered, and the judgment of the attending clinician. Wide variations in the needed dosage are to be expected in view of the variety of IL-6 and IL-21 antagonists available and the differing efficiencies of various routes of administration. Variations in these dosage levels can be adjusted using standard empirical routines for optimization, as is well understood in the art. Administrations can be single or multiple (e.g., 2- or 3-, 4-, 6-, 8-, 10-, 20-, 50-, 100-, 150-, or more fold). Encapsulation of the IL-6 and IL-21 antagonists in a suitable delivery vehicle (e.g., polymeric microparticles or implantable devices) may increase the efficiency of delivery.

The duration of treatment with any composition provided herein can be any length of time from as short as one day to as long as the life span of the host (e.g., many years). For example, the IL-6 and IL-21 antagonists can be administered once a week (for, for example, 4 weeks to many months or years); once a month (for, for example, three to twelve months or for many years); or once a year for a period of 5 years, ten years, or longer. It is also noted that the frequency of treatment can be variable. For example, the present peptides can be administered once (or twice, three times, etc.) daily, weekly, monthly, or yearly.

The particular dosage of a pharmaceutical composition to be administered to the patient will depend on a variety of considerations including the nature of the disease or condition (e.g., the extent of compatibility of a transplant or the severity of an autoimmune disease), the schedule of administration, the age and physical characteristics of the patient, and other considerations known to those of ordinary skill in the art. Dosages can be established using clinical approaches known in the art. It is presently believed that dosages in the range of 0.1 to 100 mg of antagonist (e.g., peptide antagonist) per kilogram of the patient's body weight will be useful, and a range of 1 to 100 mg per kg is generally preferred, particularly where administration is by injection or ingestion. Topical dosages may utilize formulations containing generally as low as 0.1 mg of peptide per ml of liquid carrier or excipient, with multiple applications being made as necessary. In some embodiments, the dosages can be in the range of 0.01-1,000 μg/kg.

The antagonists described herein can be administered in “therapeutically effective amounts,” in which case they are administered in amounts that are or are expected to be effective, either upon a single- or multiple-dose administration to a patient, in curing, reducing the severity of, or ameliorating one or more symptoms of a condition or disorder described herein (e.g., graft-versus-host disease or an autoimmune disease). A therapeutically effective amount is an amount that brings about or is expected to bring about a clinically beneficial outcome (e.g., the prolonged survival of a graft or the survival of a graft without the need for immunosuppression).

While the present methods clearly contemplate the treatment and prevention of graft rejection and autoimmune disease in human patients, the invention is not so limited. Veterinary uses are also within the scope of the present invention. Accordingly, the present methods can be applied to treat mammalian and avian subjects.

Conditions amenable to treatment: As noted above, the present compositions and methods can be used in any instance in which a host receives a graft of biological material other than an autograft. The graft can be the transplant of a heart, kidney, liver, lung, pancreas, bone marrow, cartilage, skin, cornea, neuronal tissue, muscle, or of tissues, portions or cells of these organs (e.g., a lobe of a lung or liver). While the methods can be limited to administration of an IL-6 and IL-21 antagonist to the recipient of the transplant, they also encompass administration of an IL-6 antagonist to the graft per se (e.g., before or at the time of transplant). For example, the graft can be exposed to an IL-6 antagonist prior to harvesting or prior to implantation into the recipient. The recipient can be treated with an IL-21 antagonist or with both an IL-6- and an IL-21 antagonist.

Autoimmune diseases can be treated as described herein with an IL-6 antagonist and an IL-21 antagonist. Autoimmune diseases amenable to treatment include diabetes mellitus (e.g., type I diabetes); multiple sclerosis; an arthritic disorder (e.g., rheumatoid arthritis (RA), juvenile rheumatoid arthritis, osteoarthritis, psoriatic arthritis, or ankylosing spondylitis (preferably, RA)); myasthenia gravis; vasculitis; systemic lupus erythematosus (SLE); glomerulonephritis; autoimmune thyroiditis; a skin inflammatory disorder (e.g., dermatitis (including atopic dermatitis and eczematous dermatitis), scleroderma, or psoriasis); lupus erythematosus; a fibrosis or fibrotic disorder (e.g., pulmonary fibrosis or liver fibrosis); a respiratory disorder (e.g., asthma or COPD); an atopic disorder (e.g., including allergy); or an intestinal inflammatory disorder (e.g., an IBD such as Crohn's disease or ulcerative colitis). The compositions and methods of the invention can also be useful for diseases in which ischemia reperfusion or anoxia-hypoxia occur including acute kidney injury (also known as acute tubular necrosis) or myocardial infarct.

Other conditions amenable to treatment include cancers in which IL-6 is expressed or overexpressed (e.g., myeloma/plasmacytoma), post-menopausal osteoporosis and cancer cachexy. We may refer to treatable conditions other than autoimmune diseases as immune cell-associated pathologies. These pathologies may be associated with aberrant activity of one or more mature T cells (mature CD8⁺, mature CD4⁺ T-cells), mature NK cells, B cells, macrophages and megakaryocytes).

The present methods can include a step of identifying a patient in need of treatment as described herein.

Any method known to one of ordinary skill in the art can be used to determine if a particular response is induced. Clinical methods that can assess the degree of a particular disease state can be used to determine if a response is induced. For example, in a transplant patient, clinical methods can include blood tests to assess organ function, ultrasound analysis of the size of the transplanted organ and blood flow, x-rays, biopsies, electrocardiograms and echocardiograms to monitor heart function, pulmonary function tests, molecular analysis such as AlloMap™ to monitor the activity of specific genes in white blood cells to determine the risk of acute cellular rejection. The particular methods used to evaluate a response will depend upon the nature of the patient's disorder, the patient's age, and sex, other drugs being administered, and the judgment of the attending clinician.

The IL-6 antagonists and the IL-21 antagonists provided herein can be administered in conjunction with other therapeutic modalities to an individual in need of therapy. The present polypeptides can be given prior to, simultaneously with or after treatment with other agents or regimes. For example, the IL-6 antagonists and the IL-21 antagonists can be administered in conjunction with standard therapies used on organ transplantation, such as rapamycin or an immunosuppressant, e.g., Azathioprine, Basiliximab, Cyclosporine, Daclizumab, Muromonab-CD3, Mycophenolic Acid, Mycophenolate Mofetil, Prednisone, Sirolimus or Tacrolimus. In addition to immunosuppressant drugs, other medications may include: antibiotics, anti-fungal medications, anti-ulcer medications, diuretics, antivirals or statins.

Similarly, when the IL-6 antagonists and the IL-21 antagonists are used for treatment of autoimmune diseases or other immune cell-associated pathologies, they can be administered along with standard treatments, e.g., chemotherapy, for such disorders.

### EXAMPLES

We have successfully bred IL-6 knockout (KO) and IL-21 receptor (IL-21R) KO mice into both C57BL/6 and Balb/c backgrounds and also crossed them with C57BL/6 and Balb/c foxp3gfp knockin mice enabling precise tracking of Foxp3⁺Tregs based on their expression of green fluorescent protein. These genetically altered mice are within the scope of the present invention; they are an aspect of the invention. While the IL-6 blocking antibodies are available commercially, we developed the mutant antagonist-type IL-21.Ig. This antagonist is also within the scope of the present invention; it is an aspect of the invention. The mutant of IL-21 (mIL-21) binds to the cytokine-specific α subunit, but not to the common γ-chain component of the IL-21 receptor complex, and thus fails to activate down-stream JAK-STAT signaling. As noted above, IL-21 consists of 131 amino acids that form a four-helix bundle and exhibit homology with IL-2, IL-4, IL-7 and IL-15. The antagonist, mutant of IL-21 (mIL-21), has two structurally conservative mutations on the 4^{th} helix (substitution Gln→Ala), allowing its binding to the cytokine-specific α subunit, but not the common γ-chain, thus precluding effective signal transduction upon the ligation of the IL-21 receptor; moreover, the mutant IL-21 polypeptide is joined to the Fc region of murine IgG2a (“mIL-21/Fc”, and the Fc region includes the hinge, CH2 and CH3 domains of murine IgG2a), which prolongs its circulating half-life and provides a potential means to kill activated, IL-21R-bearing target cells, via the activation of complement and FcR⁺ leukocytes. The glutamine→alanine mutation in the 4^{th} helix of IL-21 produces a high affinity receptor antagonist. This mutant polypeptide competes effectively with wild type IL-21 polypeptides and can inhibit one or more of the events that normally occur in response to IL-21 signaling, such as cellular proliferation. Mutant IL-21.Ig is unlikely to be immunogenic because the mutant portion of the protein differs from the corresponding wild type protein by only a few substituted residues.

The studies described below were designed to determine whether the absence of IL-6 and of IL-21 receptors skews the allograft response toward commitment of donor reactive CD4⁺T-cells to the Treg phenotype, central to the induction and maintenance of transplant tolerance. We suggest that the simultaneous blockade of IL-6 and IL-21 will promote graft tolerance more efficiently than the blockade of only one pathway.

Our studies rely on three inter-related components: (1) cardiac transplantation in the wild-type and knock-out mice; (2) real-time comparison of gene deletions and protein therapy; and (3) investigation of immune mechanisms underlying effects of the gene deletions and actions of tested therapy. We discuss these components in turn.

Cardiac transplantation in the wild-type and knock-out mice: We chose to test our strategy in a clinically relevant model of cardiac transplantation (16) in order to maximize the translational potential of our findings. Wild-type, IL-6 KO and IL-21R KO mice are available in our laboratory on both C57BL/6 and Balb/c backgrounds and with and without additional expression of eGFP under the control of endogenous Foxp3 promoter (foxp3gfp KI). To explore the relevance of expression of IL-6 and IL-21R by the donor and recipient tissues, respectively, the outcome of following transplant combinations will be evaluated:
1. C57BL/6 (H-2b) WT donor heart→Balb/c (H-2d) WT, IL-6 KO or IL-21R KO recipient
2. C57BL/6 (H-2b) IL-6 KO donor heart→Balb/c (H-2d) WT, IL-6 KO or IL-21R KO recipient
3. C57BL/6 (H-2b) IL-21R KO donor heart→Balb/c (H-2d) WT, IL-6 KO or IL-21R KO recipient

Five to ten transplants in each group will be performed in the initial phase of the project to define the site (donor, recipient, or both) and the type (IL-6, IL-21R) of gene ablation with the optimal potential to prolong cardiac allograft survival. The total number of transplants performed in one group during the time course of the project will depend on the presence, or absence of pre-existing preliminary data in the given transplant combination, with our final goal to obtain graft survival data from 10 animals within each group (totaling 90 transplants). In all transplant models, the survival of cardiac allografts will be assessed daily by palpation through the abdominal wall (17). Clinical heart rejection will be diagnosed upon cessation of the heartbeat. Heart allograft survival will be analyzed by the Kaplan-Meier test. Although palpation of cardiac action is informative as to graft survival, a more detailed study will eventually be warranted in the models prone to the development of transplant tolerance. Doppler-based sonography can be used to precisely determine remaining cardiac function in our models.

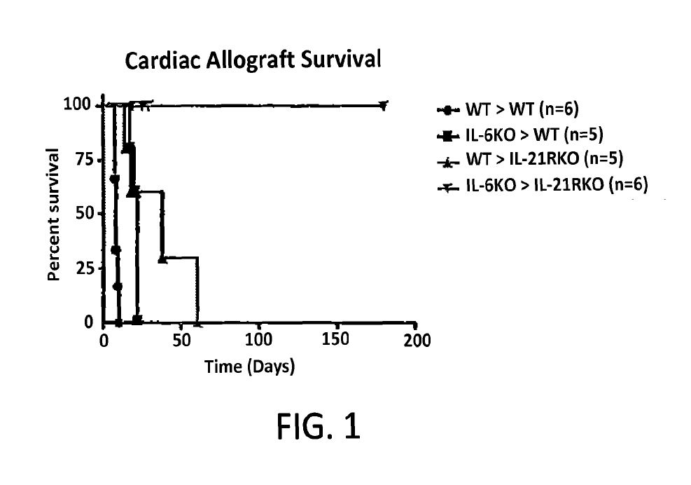

To date we have performed several preliminary experiments. Transplantation of C57BL/6 cardiac grafts (H-2b) into syngeneic C57BL/6 recipients (n=3), as well as transplantation of Balb/c cardiac grafts (H-2d) into Balb/c recipients (n=3) resulted in long-term graft survival (>150 days). Fully MHC mismatched C57BL/6 (H-2b) heart allografts underwent acute allograft rejection within 9 days (n=6) after transplantation into wild type (WT) Balb/c (H-2d) recipients. In contrast, IL-6-deficient C57BL/6 heart allografts survived up to 19 days when transplanted into WT Balb/c recipients (n=5). Our data also suggest that a deficiency of IL-21R in the recipient results in a longer graft survival (median survival time of 38 days) when compared to the effect of IL-6 deficiency in the donor (MST=19 days). Finally, we observe that a long-term, drug-free acceptance (>180 days) of cardiac allografts can be achieved in the absence of donor IL-6 and recipient IL-21R, although only 3 transplants have been followed thus far. Three more recipients are at earlier stages post-transplant, and similarly, are rejection-free. Our pilot graft survival results are summarized in FIG. 1.

After locating and determining the combination of gene ablation resulting in optimal graft protection, additional transplants will be performed in the select groups to elucidate mechanisms through which cytokine/cytokine receptor deficiency affects allograft survival. Our preliminary data suggest a potent, synergistic effect of the combined blockade.

Real-time comparing outcome of gene deletions and protein therapy: We propose a system that will attempt to replicate the most outstanding findings obtained in the gene-knock out models by short-term administration of protein therapies, targeting the gene product in the wild-type animals. We hypothesize that the outcome of IL-6, IL-21R, or combined IL-6/IL-21R therapeutic blockade will be similar to the one obtained in the gene knock-out models. This will be tested by intraperitoneal administration of the following agents as monotherapies or in combination to treat transplant recipients:
1. neutralizing anti-IL-6 antibody (clone MP5-20F3, BioXCell, New Lebanon, N.H.), 3×0.5 mg on days 0, 2 and 4 post-Tx,
2. mutant antagonist-type IL-21.Ig, 8×5 μg on days 0-7 post-Tx.

The dosing of the reagents may be optimized during the time-course of the project in order to achieve the maximal graft survival. Based on our previous experience with the fusion proteins, several doses are required to saturate available receptors in vivo, in order to achieve stable and measurable circulating levels. We are proposing 8 doses of mutant IL-21.Ig during the first week post-transplant to achieve therapeutic levels. While anti-IL-6 antibodies are commercially available, the mutant IL-21.Ig which acts as a stereospecific blocker of the IL-21R has been produced using both transient transfection and stable cell lines.

We suspect that the ascendancy of graft-protective anti-donor immunity will endure following the withdrawal of therapy and tolerance will thereby be induced. Should tolerance not be obtained we would first increase the duration of therapy two fold. Again we suspect that this will produce tolerance, but should this not be the case, we will add low, non-tolerizing doses of rapamycin because mTOR blockade synergizes with tolerance promoting strategies by adding AICD of effector, not regulatory, T-cells (18) and supporting commitment of naïve T cells into the regulatory T-cell phenotype (10).

Investigation of immune mechanisms underlying effects of the gene deletions and actions of tested therapy: To gain mechanistic insight as to the impact of loss of IL-6 and IL-21 driven responses, we will analyze the allograft response at the tissue (allograft), cellular (flow cytometry) and molecular (RT-PCR) levels, comparing transplant models with the earlier onset of rejection and models prone to variable degrees of graft acceptance. The comparison will be made at the time point (usually MST), at which rejection occurs in the control group (e.g. comparing WT→WT with IL-6 KO→IL-21R KO on day 8 post-Tx, or comparing WT→IL-21R KO with IL-6 KO→IL-21R KO on day 38). We will evaluate the following parameters.

We will assess graft histopathology to assess tissue structure, integrity and the nature (surface and intracellular markers) of cellular infiltrate in the wild-type and gene knock-out transplant models. Hematoxylin-eosin and immunofluorescene staining will be performed on the explanted heart allografts according to methods previously published and known in the art. Several pilot studies have been performed thus far. We have compared the tissue histology of the heart explanted from wild-type donor, WT C57BL/6 heart undergoing acute rejection in the WT Balb/c recipient, day 8 post-treatment and the C57BL/6 IL-6 KO heart demonstrating long-term acceptance in the IL-21R KO Balb/c recipient, on day 100 post-transplant.

In order to gain further information on the nature of allograft infiltrate, immunofluorescence staining (CD4, Foxp3) was performed. When comparing WT→WT and IL-6 KO→WT combination on day 7 post-transplant, increased frequency of CD4 and Foxp3-expressing cells is apparent in the IL-6-deficient grafts. Immunofluorescence staining for CD45 (a pan-leukocyte marker), CD8, NKp46 (a universal NK-cell marker), IL-17A, IFNγ and granzyme B will be included in the further investigations to determine pattern of graft infiltration by major immune subsets. Consistent with our preliminary studies, we anticipate increased proportion of Foxp3⁺Tregs in the IL-6 KO and IL-21R KO models, possibly accompanied by decreased inflammatory (Th1, Th17 cells and cytotoxic CD8) infiltration of heart allografts.

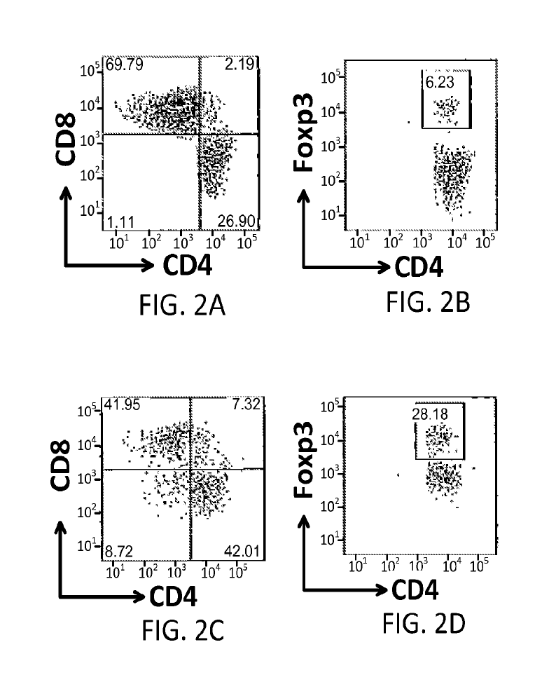

Immune cell responses will be assessed to test the hypothesis that allograft response conducted in the absence of IL-6 and IL21R tilts toward the graft-protective Treg immunity. Using multi-color flow cytometry, we will evaluate the frequency and phenotype of major immune subsets (CD4, CD8, NK cells, NKT cells, B cells) infiltrating allograft (following their isolation using collagenase-based protocol), and those present in the regional, graft-draining lymph nodes. The nature of immune response will be determined using intracellular staining for lineage-specific transcription factors (Foxp3, Tbet, GATA3, RORγt), cytokines (IFNγ, IL-17A, IL-4, TGFβ, IL-10) and activation markers (CD69, CD44), according to the previously published methods long used in Dr. Koulmanda and Strom's laboratories (11, 12, 19). We anticipate a central role for the enduring functional supremacy of Foxp3-expressing Tregs, induced or naturally occurring, over cytopathic T-cells in the immune mechanisms underlying prolonged allograft survival in IL-6 KO and IL-21R KO transplant models, i.e. increased ratio of Foxp3⁺Tregs to effector T-cells (Th1, Th17, CTL) in the IL-6 KO to the WT and IL-6 KO to the IL-21R KO models compared to the WT transplant models. We also anticipate the numerical dominance of graft-infiltrating effector T-cells over Treg cells during the whole post-transplant period in the WT C57BL/6 to the WT Balb/c model, while proposing the possibility of a sustained Treg dominance in the IL-6 KO to the IL-21R KO model, underlying immune tolerance. While primarily altering cytokine milieu of alloresponse, rather than targeting specific immune subsets, a broader extent of graft-protective modulation, possibly involving CD8, NK and NKT cells (expressing IL-21R) is anticipated and will be investigated. Similarly to the gene-knock-out models, we expect to provide the evidence that treatment with anti-IL-6 and/or mutant IL-21.Ig favors graft protective versus graft-destructive immunity. Our preliminary study, performed on the heart infiltrating cells isolated from C57BL/6 WT heart transplanted in the Balb/c WT recipient (FIG. 2A and FIG. 2B) and IL-6K° heart in the WT recipient (FIG. 2C and FIG. 2D) on day 7 post-treatment, suggests increased CD4/CD8 T-cell ratio and increased proportion of Foxp3(GFP)-expressing T-cells in the absence of donor-derived IL-6.

Gene expression profiling will be carried out using Real-Time PCR to assess molecular signature of the grafts and immune subsets prone to the rejection or variable degrees of tolerance. We will evaluate the intra-graft expression of pro-inflammatory (IL-1β, IL-6, TNF-α) and anti-inflammatory (IL-10, TGFβ, IL-1RA) cytokines and T-cell lineage markers (IL-2, IFNγ, Tbet, GATA-3, IL-4, IL-5, IL-17A, RORγ, IL-23R, IL-10, Foxp3, CTLA-4, CD39) in explanted heart allografts. An immune subset-focused investigations (CD4, CD8, NK, NKT) are feasible following cell sorting of the defined populations from heart infiltrate or lymph nodes. We anticipate decreased intra-graft expression of pro-inflammatory cytokines, increased expression of anti-inflammatory cytokines and increased expression of Treg-specific markers in the IL-6 KO and IL-21R KO transplant models. Gene transcript levels in tissues and cells harvested from control and gene-knock-out/treated animals will be compared using methods previously published by our group (11, 12, 19).

Statistical Methods: All data will be analyzed using the statistical software Prism (GraphPad, San Diego, Calif.). All results will be additionally validated in the consult with Beth Israel Deaconess Hospital Statistical Core.

### Example 2

### IL-6 Promotes Accelerated Islet Allograft Rejection by Destroying Both nTreg and iTreg Compartments and Tilting the Balance Between Treg/TH17

In Vivo Imaging.

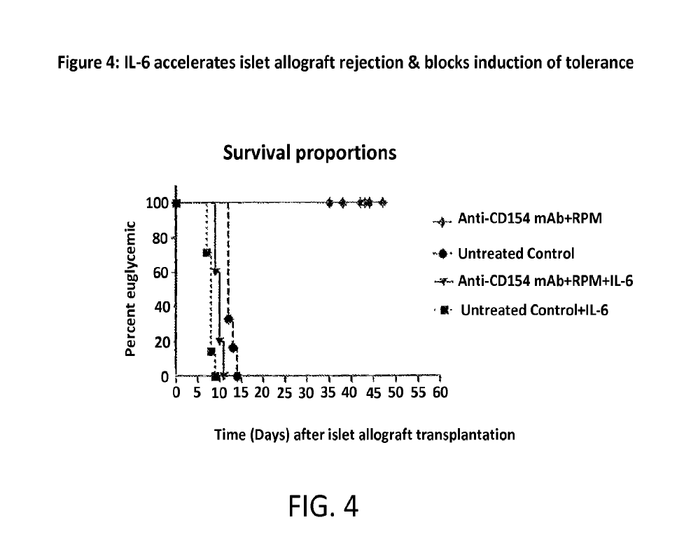

We have developed a “color-coded” adoptive transfer model for T cell subset identification that enables serial analysis of islet allograft infiltrating yellow induced regulatory T cells (iTreg), green natural regulatory T cells (nTreg) and red effector T cells (Teff) in live animals using endoscopic confocal microscopy (Nat. Med. 2010). Using this model, we found that delivery of IL-6 in this model via osmotic pumps promoted accelerated allograft rejection (FIG. 4,5). The IL-6 triggered accelerated allograft rejection was associated with inhibition of conversion of naive CD4+ T cells to iTreg as well as the loss of nTreg phenotype. Flow cytometry analysis based on incorporation of CD45.1 congenic marker into the model demonstrated that the majority of nTreg converted to either Th17 or Th1 phenotype post allograft transplant when IL-6 osmotic pump was present. Additionally, the accelerated rejection could not be prevented by anti-CD154 mAb plus Rapamycin, a strong tolerizing regimen that promotes iTreg conversion and nTreg stability in hosts not receiving IL-6 treatment (FIG. 4, 5). The data provided new texture as to the importance of the integrity of both iTregs and nTregs in achieving allograft tolerance. Furthermore, the data suggested the balance between Treg and aggressive Teff (Th17/Th1) may be an essential indicator prognosing allograft outcome. Experiments based on Foxp3 RFP-RorγT eGFP double indicator whole mice confirmed that the balance between Treg and TH17 cells was tilted upon IL-6 infusion, which was also associated with accelerated rejection. The data supports the idea that selective anti-inflammatory treatment may be imperative for induction and maintenance of allograft tolerance, especially in complex clinical situations where inflammation plays a significant role.

### Example 3

### Histology

We evaluated cardiac allograft tissue using tissue injury markers hematoxylin and eosin stain (“H&E”), Caspase 3,4-hydroxynonenal (membrane lipid peroxidation) and 8-Hydroxy-2′-deoxyguanosine (DNA damage). We monitored immune cell infiltrates using the following markers: CD4, CD8, CD11b, Foxp3 and NKp46. We will also monitor immune call infiltrates using CD45, CD3, RORgt, IL-17A, Tbet, GrzB, CD11b, CD69, CD11c, CD44, Gr1, F4/80, B220 and TIM4.

A number of embodiments of the invention have been described. Nevertheless, it will be understood that various modifications may be made without departing from the spirit and scope of the invention. Accordingly, other embodiments are within the scope of the following claims.

1. A bi-specific immunoglobulin comprising a first portion that specifically binds an IL-6 or an IL-6 receptor and a second portion that specifically binds an IL-21 or an IL-21 receptor.

2. The bispecific immunoglobulin of claim 1, wherein the first portion specifically binds an IL-6 and the second portion specifically binds an IL-21; the first portion specifically binds an IL-6 receptor and the second portion specifically binds an IL-21 receptor; the first portion specifically binds an IL-6 and the second portion specifically binds an IL-21 receptor; or the first portion specifically binds an IL-6 receptor and the second portion specifically binds an IL-21.

3. The bi-specific immunoglobulin of claim 1, wherein the antibody is a bi-specific monoclonal antibody or a biologically active variant thereof, a chemically linked F(ab′)₂ or a biologically active variant thereof, or a bi-specific T cell engager or a biologically active variant thereof.

4-5. (canceled)

6. The bi-specific immunoglobulin of claim 1, wherein the immunoglobulin further comprises a toxin or radioisotope.

7. The bi-specific immunoglobulin of claim 1, wherein the immunoglobulin further comprises a detectable label.

8. The bi-specific immunoglobulin of claim 7, wherein the detectable label is used in performing positron-emission tomography (PET); is used to perform SPECT imaging; is used in magnetic resonance imaging; is detectable by X-ray; or is detectable by ultrasound.

9-35. (canceled)

36. A pharmaceutical composition comprising first and second agents, wherein the first agent comprises an IL-6 pathway antagonist and the second agent comprises an IL-21 pathway antagonist.

37. The pharmaceutical composition of claim 36, wherein the first agent comprises an anti-IL-6 antibody or a biologically active variant thereof, an anti-IL-6 receptor antibody or a biologically active variant thereof, a mutant IL-6, a soluble IL-6 receptor, optionally coupled to an immunoglobulin, or a small organic compound that blocks IL-6 or an IL-6 receptor.

38. The pharmaceutical composition of claim 37, wherein the mutant IL-6 binds but does not activate the corresponding IL-6 receptor.

39. The pharmaceutical composition of claim 37, wherein the soluble IL-6 receptor binds a corresponding IL-6.

40. The pharmaceutical composition of claim 36, wherein the first agent further comprises a toxin, a radioisotope, or detectable label.

41. The pharmaceutical composition of claim 36, wherein the second agent comprises an anti-IL-21 antibody or a biologically active variant thereof, an anti-IL-21 receptor antibody or a biologically active variant thereof, a mutant IL-21, a soluble IL-21 receptor, optionally coupled to an immunoglobulin, or a small organic compound that blocks IL-21 or an IL-21 receptor.

42. The pharmaceutical composition of claim 41, wherein the mutant IL-21 binds but does not activate the corresponding IL-21 receptor.

43. The pharmaceutical composition of claim 41, wherein the soluble IL-21 receptor binds a corresponding IL-21.

44. The pharmaceutical composition of claim 41, wherein the second agent further comprises a toxin, a radioisotope, or detectable label.

45. A method of reducing the likelihood of graft rejection in a patient, the method comprising administering to the patient a therapeutically effective amount of the pharmaceutical composition comprising first and second agents, wherein the first agent comprises an IL-6 pathway antagonist and the second agent comprises an IL-21 pathway antagonist or a bi-specific immunoglobulin comprising a first portion that specifically binds an IL-6 or an IL-6 receptor and a second portion that specifically binds an IL-21 or an IL-21 receptor.

46-47. (canceled)

48. The method of claim 45, wherein the graft is an allograft or xenograft.

49. The method of claim 45, wherein the graft is an organ graft.

50. (canceled)

51. The method of claim 48, wherein the graft comprises a population of cells that do not define an intact organ.

52. The method of claim 51, wherein the population of cells comprises stem cells.

53-65. (canceled)


SEQUENCE LISTING (43 sequences)

SEQ ID NO: 1 - protein, 162 aa, Homo sapiens
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 2 - protein, 162 aa, Homo sapiens; identical to SEQ ID NO: 1

SEQ ID NO: 3 - protein, 20 aa, Homo sapiens
ESSKEALAENNLNLPKMAEK

SEQ ID NO: 4 - protein, 11 aa, Homo sapiens
GALAENNLNLP

SEQ ID NO: 5 - protein, 11 aa, Homo sapiens
ESSKEALAENN

SEQ ID NO: 6 - protein, 10 aa, Homo sapiens
NLNLPKMAEK

SEQ ID NO: 7 - protein, 21 aa, Homo sapiens
RHVVQLRAQEEFGQGEWSEWS

SEQ ID NO: 8 - protein, 12 aa, Homo sapiens
RHVVQLRAQEEF

SEQ ID NO: 9 - protein, 13 aa, Homo sapiens
GQLRAQEEFGQGE

SEQ ID NO: 10 - protein, 11 aa, Homo sapiens
EFGQGEWSEWS

SEQ ID NO: 11 - protein, 18 aa, Homo sapiens
AGSHPSRWAGMGRRLLLR

SEQ ID NO: 12 - protein, 11 aa, Homo sapiens
HPSRWAGMGRR

SEQ ID NO: 13 - protein, 10 aa, Homo sapiens
AGSHPSRWAG

SEQ ID NO: 14 - protein, 13 aa, Homo sapiens
CGHPSRWAGMGRR

SEQ ID NO: 15 - protein, 10 aa, Homo sapiens
GMGRRLLLRS

SEQ ID NO: 16 - protein, 17 aa, Homo sapiens
MVKDLQHHCVIHDAWSG

SEQ ID NO: 17 - protein, 14 aa, Homo sapiens
MVKDLQHHCVIHDA

SEQ ID NO: 18 - protein, 24 aa, Homo sapiens
RLFQHSPAGDFQEPCQYSQESQLF

SEQ ID NO: 19 - protein, 12 aa, Homo sapiens
EPSQYSQESQLF

SEQ ID NO: 20 - protein, 13 aa, Homo sapiens
AGDFQEPSQYSQE

SEQ ID NO: 21 - protein, 24 aa, Homo sapiens
RLFQNSPAGDFQEPCQYSQESQLF

SEQ ID NO: 22 - protein, 15 aa, Homo sapiens
KTSMHPPYSLGQLVP

SEQ ID NO: 23 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 24 - protein, 468 aa, Homo sapiens
MLAVGCALLAALLAAPGAALAPRRCPAQEVARGVLTSLPGDSVTLTCPGVEPEDNATVHWVLRKPAAGSHPSRWAGMGRRLLLRSVQLHDSGNYSCYRAGRPAGTVHLLVDVPPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTESRSPPAENEVSTPMQALTTNKDDDNILFRDSANATSLPVQDSSSVPLPTFLVAGGSLAFGTLLCIAIVLRFKKTWKLRALKEGKTSMHPPYSLGQLVPERPRPTPVLVPLISPPVSPSSLGSDNTSSHNRPDARDPRSPYDISNTDYFFPR

SEQ ID NO: 25 - DNA, 636 bp, Homo sapiens
gctgaagtgaaaacgagaccaaggtctagctctactgttggtacttatgagatccagtcctggcaacatggagaggattgtcatctgtctgatggtcatcttcttggggacactggtccacaaatcaagctcccaaggtcaagatcgccacatgattagaatgcgtcaacttatagatattgttgatcagctgaaaaattatgtgaatgacttggtccctgaatttctgccagctccagaagatgtagagacaaactgtgagtggtcagctttttcctgctttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagaattcctagaaagattcaaatcacttctccaaaagatgattcatcagcatctgtcctctagaacacacggaagtgaagattcctgaggatctaacttgcagttggacactatgttacatactctaatatagtagtgaaagtcatttctttgtattccaagtggaggagtacaatatattagcgatgg

SEQ ID NO: 26 - DNA, 617 bp, Homo sapiens
gctgaagtgaaaacgagaccaaggtctagctctactgttggtacttatgagatccagtcctggcaacatggagaggattgtcatctgtctgatggtcatcttcttggggacactggtccacaaatcaagctcccaaggtcaagatcgccacatgattagaatgcgtcaacttatagatattgttgatcagctgaaaaattatgtgaatgacttggtccctgaatttctgccagctccagaagatgtagagacaaactgtgagtggtcagctttttcctgctttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagaattcctagaaagattcaaatcacttctccaaaagatgattcatcagcatctgtcctctagaacacacggaagtgaagattcctgaggatctaacttgcagttggacactatgttacatactctaatatagtagtgaaagtcatttctttgtattccaagtggaggag

SEQ ID NO: 27 - protein, 16 aa, Homo sapiens
MIHQHLSSRTHGSEDS

SEQ ID NO: 28 - protein, 7 aa, Homo sapiens
VSTLSFI

SEQ ID NO: 29 - protein, 153 aa, Homo sapiens
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKVSTLSFI

SEQ ID NO: 30 - protein, 162 aa, Homo sapiens; identical to SEQ ID NO: 1

SEQ ID NO: 31 - protein, 162 aa, Homo sapiens; identical to SEQ ID NO: 1

SEQ ID NO: 32 - protein, 162 aa, Homo sapiens; identical to SEQ ID NO: 1

SEQ ID NO: 33 - protein, 162 aa, Homo sapiens; identical to SEQ ID NO: 1

SEQ ID NO: 34 - protein, 162 aa, Homo sapiens; identical to SEQ ID NO: 1

SEQ ID NO: 35 - protein, 162 aa, Homo sapiens; identical to SEQ ID NO: 1

SEQ ID NO: 36 - DNA, 616 bp, Homo sapiens
ctgaagtgaaaacgagaccaaggtctagctctactgttggtacttatgagatccagtcctggcaacatggagaggattgtcatctgtctgatggtcatcttcttggggacactggtccacaaatcaagctcccaaggtcaagatcgccacatgattagaatgcgtcaacttatagatattgttgatcagctgaaaaattatgtgaatgacttggtccctgaatttctgccagctccagaagatgtagagacaaactgtgagtggtcagctttttcctgctttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagaattcctagaaagattcaaatcacttctccaaaagatgattcatcagcatctgtcctctagaacacacggaagtgaagattcctgaggatctaacttgcagttggacactatgttacatactctaatatagtagtgaaagtcatttctttgtattccaagtggaggag

SEQ ID NO: 37 - DNA, 706 bp, Homo sapiens
ctgaagtgaaaacgagaccaaggtctagctctactgttggtacttatgagatccagtcctggcaacatggagaggattgtcatctgtctgatggtcatcttcttggggacactggtccacaaatcaagctcccaaggtcaagatcgccacatgattagaatgcgtcaacttatagatattgttgatcagctgaaaaattatgtgaatgacttggtccctgaatttctgccagctccagaagatgtagagacaaactgtgagtggtcagctttttcctgctttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagaattcctagaaagattcaaatcacttctccaaaaggtatctaccttaagtttcatttgattttctgctttatctttacctatccagatttgcttcttagttactcacggtatactatttccacagatgattcatcagcatctgtcctctagaacacacggaagtgaagattcctgaggatctaacttgcagttggacactatgttacatactctaatatagtagtgaaagtcatttctttgtattccaagtggaggag

SEQ ID NO: 38 - protein, 538 aa, Homo sapiens
MPRGWAAPLLLLLLQGGWGCPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKEGWNPHLLLLLLLVIVFIPAFWSLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 39 - protein, 538 aa, Homo sapiens; identical to SEQ ID NO: 38

SEQ ID NO: 40 - protein, 560 aa, Homo sapiens
MSCRCIFLMKHGERVGWPVGVSMPRGWAAPLLLLLLQGGWGCPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKEGWNPHLLLLLLLVIVFIPAFWSLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 41 - protein, 15 aa, Artificial Sequence
  Description of Artificial Sequence Synthetic peptide
GGGGSGGGGSGGGGS

SEQ ID NO: 42 - protein, 162 aa, Artificial Sequence
  Description of Artificial Sequence Synthetic polypeptide
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLAKMIHQHLSSRTHGSEDS

SEQ ID NO: 43 - protein, 162 aa, Artificial Sequence
  Description of Artificial Sequence Synthetic polypeptide
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHAHLSSRTHGSEDS